SEQUENCE LISTING (87 sequences)

SEQ ID NO: 1 - RNA, 28 nt, synthetic construct
  source 1..28: mol_type = other RNA
caccaggcggccgcgcacgtcctccatg

SEQ ID NO: 2 - RNA, 24 nt, synthetic construct
  source 1..24: mol_type = other RNA
ccaggcggccgcgcacgtcctcca

SEQ ID NO: 3 - RNA, 24 nt, synthetic construct
  source 1..24: mol_type = other RNA
ggccgcgcacgtcctccatgtccg

SEQ ID NO: 4 - RNA, 24 nt, synthetic construct
  source 1..24: mol_type = other RNA
tgcaccaggcggccgcgcacgtcc

SEQ ID NO: 5 - RNA, 24 nt, synthetic construct
  source 1..24: mol_type = other RNA
actgcaccaggcggccgcgcacgt

SEQ ID NO: 6 - RNA, 24 nt, synthetic construct
  source 1..24: mol_type = other RNA
gtactgcaccaggcggccgcgcac

SEQ ID NO: 7 - RNA, 22 nt, synthetic construct
  source 1..22: mol_type = other RNA
ccgcgcacgtcctccatgtccg

SEQ ID NO: 8 - RNA, 24 nt, synthetic construct
  source 1..24: mol_type = other RNA
cgcgcacgtcctccatgtccgcgc

SEQ ID NO: 9 - RNA, 24 nt, synthetic construct
  source 1..24: mol_type = other RNA
tcaggcggccgcgcacgtcctcca

SEQ ID NO: 10 - RNA, 24 nt, synthetic construct
  source 1..24: mol_type = other RNA
ctaggcggccgcgcacgtcctcca

SEQ ID NO: 11 - RNA, 24 nt, synthetic construct
  source 1..24: mol_type = other RNA
cctggcggccgcgcacgtcctcca

SEQ ID NO: 12 - RNA, 24 nt, synthetic construct
  source 1..24: mol_type = other RNA
ccatgcggccgcgcacgtcctcca

SEQ ID NO: 13 - RNA, 24 nt, synthetic construct
  source 1..24: mol_type = other RNA
ccagtcggccgcgcacgtcctcca

SEQ ID NO: 14 - RNA, 24 nt, synthetic construct
  source 1..24: mol_type = other RNA
ccaggtggccgcgcacgtcctcca

SEQ ID NO: 15 - RNA, 24 nt, synthetic construct
  source 1..24: mol_type = other RNA
ccaggctgccgcgcacgtcctcca

SEQ ID NO: 16 - RNA, 24 nt, synthetic construct
  source 1..24: mol_type = other RNA
ccaggcgtccgcgcacgtcctcca

SEQ ID NO: 17 - RNA, 24 nt, synthetic construct
  source 1..24: mol_type = other RNA
ccaggcggtcgcgcacgtcctcca

SEQ ID NO: 18 - RNA, 24 nt, synthetic construct
  source 1..24: mol_type = other RNA
ccaggcggctgcgcacgtcctcca

SEQ ID NO: 19 - RNA, 24 nt, synthetic construct
  source 1..24: mol_type = other RNA
ccaggcggcctcgcacgtcctcca

SEQ ID NO: 20 - RNA, 24 nt, synthetic construct
  source 1..24: mol_type = other RNA
ccaggcggccgtgcacgtcctcca

SEQ ID NO: 21 - RNA, 24 nt, synthetic construct
  source 1..24: mol_type = other RNA
ccaggcggccgcgtacgtcctcca

SEQ ID NO: 22 - RNA, 24 nt, synthetic construct
  source 1..24: mol_type = other RNA
ccaggcggccgcgctcgtcctcca

SEQ ID NO: 23 - RNA, 24 nt, synthetic construct
  source 1..24: mol_type = other RNA
ccaggcggccgcgcatgtcctcca

SEQ ID NO: 24 - RNA, 24 nt, synthetic construct
  source 1..24: mol_type = other RNA
ccaggcggccgcgcacttcctcca

SEQ ID NO: 25 - RNA, 24 nt, synthetic construct
  source 1..24: mol_type = other RNA
ccaggcggccgcgcacgacctcca

SEQ ID NO: 26 - RNA, 24 nt, synthetic construct
  source 1..24: mol_type = other RNA
ccaggcggccgcgcacgttctcca

SEQ ID NO: 27 - RNA, 24 nt, synthetic construct
  source 1..24: mol_type = other RNA
ccaggcggccgcgcacgtcttcca

SEQ ID NO: 28 - RNA, 24 nt, synthetic construct
  source 1..24: mol_type = other RNA
ccaggcggccgcgcacgtccacca

SEQ ID NO: 29 - RNA, 24 nt, synthetic construct
  source 1..24: mol_type = other RNA
ccaggcggccgcgcacgtccttca

SEQ ID NO: 30 - RNA, 24 nt, synthetic construct
  source 1..24: mol_type = other RNA
ccaggcggccgcgcacgtcctcta

SEQ ID NO: 31 - RNA, 24 nt, synthetic construct
  source 1..24: mol_type = other RNA
ccaggcggccgcgcacgtcctcct

SEQ ID NO: 32 - RNA, 24 nt, synthetic construct
  source 1..24: mol_type = other RNA
tctgcaccaggcggccgcgcacgt

SEQ ID NO: 33 - RNA, 24 nt, synthetic construct
  source 1..24: mol_type = other RNA
attgcaccaggcggccgcgcacgt

SEQ ID NO: 34 - RNA, 24 nt, synthetic construct
  source 1..24: mol_type = other RNA
acagcaccaggcggccgcgcacgt

SEQ ID NO: 35 - RNA, 24 nt, synthetic construct
  source 1..24: mol_type = other RNA
acttcaccaggcggccgcgcacgt

SEQ ID NO: 36 - RNA, 24 nt, synthetic construct
  source 1..24: mol_type = other RNA
actgtaccaggcggccgcgcacgt

SEQ ID NO: 37 - RNA, 24 nt, synthetic construct
  source 1..24: mol_type = other RNA
actgctccaggcggccgcgcacgt

SEQ ID NO: 38 - RNA, 24 nt, synthetic construct
  source 1..24: mol_type = other RNA
actgcatcaggcggccgcgcacgt

SEQ ID NO: 39 - RNA, 24 nt, synthetic construct
  source 1..24: mol_type = other RNA
actgcactaggcggccgcgcacgt

SEQ ID NO: 40 - RNA, 24 nt, synthetic construct
  source 1..24: mol_type = other RNA
actgcacctggcggccgcgcacgt

SEQ ID NO: 41 - RNA, 24 nt, synthetic construct
  source 1..24: mol_type = other RNA
actgcaccatgcggccgcgcacgt

SEQ ID NO: 42 - RNA, 24 nt, synthetic construct
  source 1..24: mol_type = other RNA
actgcaccagtcggccgcgcacgt

SEQ ID NO: 43 - RNA, 24 nt, synthetic construct
  source 1..24: mol_type = other RNA
actgcaccaggtggccgcgcacgt

SEQ ID NO: 44 - RNA, 24 nt, synthetic construct
  source 1..24: mol_type = other RNA
actgcaccaggctgccgcgcacgt

SEQ ID NO: 45 - RNA, 24 nt, synthetic construct
  source 1..24: mol_type = other RNA
actgcaccaggcgtccgcgcacgt

SEQ ID NO: 46 - RNA, 24 nt, synthetic construct
  source 1..24: mol_type = other RNA
actgcaccaggcggtcgcgcacgt

SEQ ID NO: 47 - RNA, 24 nt, synthetic construct
  source 1..24: mol_type = other RNA
actgcaccaggcggctgcgcacgt

SEQ ID NO: 48 - RNA, 24 nt, synthetic construct
  source 1..24: mol_type = other RNA
actgcaccaggcggcctcgcacgt

SEQ ID NO: 49 - RNA, 24 nt, synthetic construct
  source 1..24: mol_type = other RNA
actgcaccaggcggccgtgcacgt

SEQ ID NO: 50 - RNA, 24 nt, synthetic construct
  source 1..24: mol_type = other RNA
actgcaccaggcggccgcgtacgt

SEQ ID NO: 51 - RNA, 24 nt, synthetic construct
  source 1..24: mol_type = other RNA
actgcaccaggcggccgcgctcgt

SEQ ID NO: 52 - RNA, 24 nt, synthetic construct
  source 1..24: mol_type = other RNA
actgcaccaggcggccgcgcatgt

SEQ ID NO: 53 - RNA, 24 nt, synthetic construct
  source 1..24: mol_type = other RNA
actgcaccaggcggccgcgcactt

SEQ ID NO: 54 - RNA, 24 nt, synthetic construct
  source 1..24: mol_type = other RNA
actgcaccaggcggccgcgcacga

SEQ ID NO: 55 - RNA, 62 nt, synthetic construct
  source 1..62: mol_type = other RNA
ggcccggctgggcgcggacatggaggacgtgcgcggccgcctggtgcagtaccgcggcgagg

SEQ ID NO: 56 - RNA, 41 nt, synthetic construct
  source 1..41: mol_type = other RNA
gcgcggacatggaggacgtgcgcggccgcctggtgcagtac

SEQ ID NO: 57 - DNA, 6793 bp, synthetic construct
  source 1..6793: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgccggcagctaagaaaaagaaactggatggcagcgtcgacatgagcaaggacaagaaaaccaaggccaagagaatgggcgtgaaggccctgctggcccacggcgaggacaagctgaccatgaccaccttcggcaagggcaacagaagcaagatcgagttcaccgagggctaccacggcagagccctggagacacccaagcacttcggcatcagaggcttcgaggtgagaagaatcgacgagaacgtggacctgtgcggcgacctggaggagggcaagaccatcgaggccctgctggtgaaccccagcgagaaggtgggcgaggactacctgaagctgaagggcaccctggagaagagattcttcggcagagagttcccccacgacaacatcagaatccagctgatctacaacatcctggacatctacaagatcctgggcatgaacgtggccgacatcctgtacgccctgggcaacatgcaggacaccgagctggacatcgacatgttcggccagagcctgaacaacgaggacaacctgaaggagtgcctgaagagaatgaggccctacatgggctacttcggcgacatcttcaagatcagccccaagggcgagaacatcgccgacagagagcacaacaagaaggtgctgagatgcatcagcgtgctgagaaacgccaccgcccacgacaagcaggacgagtacccctggttcaagagcagcgacatctacgagacaaagatcttcaaggccgacatgtggaagatcatcaaggaccagtacagagagaagatcaagaaggtgaacaaggacttcctgagcaagaacgccgtgaacatggccatcctgttcgacctgctgaacgccagagacgtggagcagaagaagcagatcaccgacgagttctacagattcaccatcagaaaggacggcaagaacctgggcatgaacctggtgaagatcagagagatcatcatcgacagatacgccagcggcctgagagacaagaagcacgacccccacagacagaagatcaacgtgatcgccgacttcctgatcttcagagccctgagccagaaccagggcatcatcgacaagaccgtgagcagcctgagactgaccaaggacgaggaggagaaggaccacgtgtaccagaacgccgccgagctggtgtggggcatggtgagcaactgcctgaccccctacttcaacgaccccaagaacaagtacatcctgaagtacaaggacgccaagacccccggcgacttcgaggactggatcaccagcaagatcagcgaggacgacggcgagcccttcgtgaaggtgctgagcttcctgtgcaacttcctggagggcaaggagatcaacgagctgctgaccgcctacatccacaagttcgagtgcatccaggacttcctgaacgtgatcagcagcctgggcgagaacgtgcagttccagcccagattcgccctgttcaacaacgccagcttcgcccagaacgtggccgtgcagctgagaatcctggccagcatcggcaagatgaagcccgacctgaccgaggccaagaggcccctgtacaaggccgccatcagaatgctgtgcccccccgagaagtgggagaagtacaccagcgacgagtggctggagaagaacatgctgctgaacagcgaggacagaaagaacgacaagaagaagaagcaggtgaaccccttcagaaacttcatcgccggcaacgtgatcgagagcagaagattcatgtacctggtgagatacagcaagcccaaggccgtgagagccatcatgcagaacagaagcatcgtgaactacgtgctgcacagactgcccagcgagcaggtgcacagatacgccagcgtgttccccgagaacttcgccgacctggagcaggagatcgacttcctgaccaagaagctgttcgagttcagcttcgaggagctgctgcacgagaaggacgtgatcctgaacaacagcagaagccacaagcccagcctggagatcgagagactgaaggccatcaccggcctgtacctgagcgtggcctacatcgccatcaagaacatcgtgaaggccaacgccagatactacatcgccttcgccgtgttcgagagagacaaggagctggtgaaggccaaggacgccagaatccagaccaagatccccgagacagacttccccgactacttctgcctgacccagtactacctggacagagacgaggagaagaagttccccggcgaccccagagacaaggaggccttcttcgagcacctgagaaagaccaagagacacttcagcaagcagtggagagagtggctgaacgagaagatcgccgacgccaagagcagccaggccaccggcctgctgctgagagaggccagaaacgacgtggagcacctgaacgtgctgagagccatccccgactacatccaggacttcagacacggcgagaagggcgagacagccatgaacagctacttcgagctgtaccactacctgatgcagagactgatgctgaagaacaccgagctggacctgagccactggagcggctggatcatgagaagcggcagacccgacagagacttgatccagatcgccttcgtgagcctggcctacaacctgcccagatacagaaacctgaccaaggagcaccacttcgacgacaccgtgctgcagaagatcagagagaaggagagcctggacacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagataactctacaaacctgtagggttctgagacggagaccacggcaggtctcattttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 58 - DNA, 9722 bp, synthetic construct
  source 1..9722: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaataaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcgccaccctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgcgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatacccatacgatgttccagattacgctggatccgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaggcggccgcgcatgccctgcaggtgatctatcgatcggccggcccctctccctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccgggccggatatcacgcgtgatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcaatgcatacatgtgtttaaacctcgacttaattaagtcgagggtcgacggtatcgataagctcgcttcacgagatcatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 59 - DNA, 18 bp, synthetic construct
  source 1..18: mol_type = other DNA
cggacatggaggacgtgc

SEQ ID NO: 60 - DNA, 18 bp, synthetic construct
  source 1..18: mol_type = other DNA
ctggtacactgccaggcg

SEQ ID NO: 61 - DNA, 20 bp, synthetic construct
  source 1..20: mol_type = other DNA
ccatggggaaggtgaaggtc

SEQ ID NO: 62 - DNA, 21 bp, synthetic construct
  source 1..21: mol_type = other DNA
gaaggggtcattgatggcaac

SEQ ID NO: 63 - DNA, 9722 bp, synthetic construct
  source 1..9722: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaataaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgcgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcgccaccctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatacccatacgatgttccagattacgctggatccgctagcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcctaggcggccgcgcatgccctgcaggtgatctatcgatcggccggcccctctccctccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccgggccggatatcacgcgtgatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcaatgcatacatgtgtttaaacctcgacttaattaagtcgagggtcgacggtatcgataagctcgcttcacgagatcatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 64 - protein, 893 aa, synthetic construct
MSKDKKTKAKRMGVKALLAHGEDKLTMTTFGKGNRSKIEFTEGYHGRALETPKHFGIRGFEVRRIDENVDLCGDLEEGKTIEALLVNPSEKVGEDYLKLKGTLEKRFFGREFPHDNIRIQLIYNILDIYKILGMNVADILYALGNMQDTELDIDMFGQSLNNEDNLKECLKRMRPYMGYFGDIFKISPKGENIADREHNKKVLRCISVLRNATAHDKQDEYPWFKSSDIYETKIFKADMWKIIKDQYREKIKKVNKDFLSKNAVNMAILFDLLNARDVEQKKQITDEFYRFTIRKDGKNLGMNLVKIREIIIDRYASGLRDKKHDPHRQKINVIADFLIFRALSQNQGIIDKTVSSLRLTKDEEEKDHVYQNAAELVWGMVSNCLTPYFNDPKNKYILKYKDAKTPGDFEDWITSKISEDDGEPFVKVLSFLCNFLEGKEINELLTAYIHKFECIQDFLNVISSLGENVQFQPRFALFNNASFAQNVAVQLRILASIGKMKPDLTEAKRPLYKAAIRMLCPPEKWEKYTSDEWLEKNMLLNSEDRKNDKKKKQVNPFRNFIAGNVIESRRFMYLVRYSKPKAVRAIMQNRSIVNYVLHRLPSEQVHRYASVFPENFADLEQEIDFLTKKLFEFSFEELLHEKDVILNNSRSHKPSLEIERLKAITGLYLSVAYIAIKNIVKANARYYIAFAVFERDKELVKAKDARIQTKIPETDFPDYFCLTQYYLDRDEEKKFPGDPRDKEAFFEHLRKTKRHFSKQWREWLNEKIADAKSSQATGLLLREARNDVEHLNVLRAIPDYIQDFRHGEKGETAMNSYFELYHYLMQRLMLKNTELDLSHWSGWIMRSGRPDRDLIQIAFVSLAYNLPRYRNLTKEHHFDDTVLQKIREKESLD

SEQ ID NO: 65 - RNA, 36 nt, synthetic construct
  source 1..36: mol_type = other RNA
ggaagataactctacaaacctgtagggttctgagac

SEQ ID NO: 66 - RNA, 36 nt, synthetic construct
  source 1..36: mol_type = other RNA
ggaagataactctacaaacctgtagagttctgagac

SEQ ID NO: 67 - DNA, 18 bp, synthetic construct
  source 1..18: mol_type = other DNA
cggacatggaggacgtgt

SEQ ID NO: 68 - RNA, 62 nt, synthetic construct
  source 1..62: mol_type = other RNA
cctcgccgcggtactgcaccaggcggccgcgcacgtcctccatgtccgcgcccagccgggcc

SEQ ID NO: 69 - RNA, 41 nt, synthetic construct
  source 1..41: mol_type = other RNA
gtactgcaccaggcggccgcgcacgtcctccatgtccgcgc

SEQ ID NO: 70 - protein, 11 aa, synthetic construct
SPKKKRKVEAS

SEQ ID NO: 71 - protein, 11 aa, synthetic construct
GPKKKRKVAAA

SEQ ID NO: 72 - protein, 7 aa, synthetic construct
PKKKRKV

SEQ ID NO: 73 - protein, 16 aa, synthetic construct
KRPAATKKAGQAKKKK

SEQ ID NO: 74 - protein, 9 aa, synthetic construct
PAAKRVKLD

SEQ ID NO: 75 - protein, 11 aa, synthetic construct
RQRRNELKRSP

SEQ ID NO: 76 - protein, 38 aa, synthetic construct
NQSSNFGPMKGGNFGGRSSGPYGGGGQYFAKPRNQGGY

SEQ ID NO: 77 - protein, 42 aa, synthetic construct
RMRIZFKNKGKDTAELRRRRVEVSVELRKAKKDEQILKRRNV

SEQ ID NO: 78 - protein, 8 aa, synthetic construct
VSRKRPRP

SEQ ID NO: 79 - protein, 8 aa, synthetic construct
PPKKARED

SEQ ID NO: 80 - protein, 8 aa, synthetic construct
POPKKKPL

SEQ ID NO: 81 - protein, 12 aa, synthetic construct
SALIKKKKKMAP

SEQ ID NO: 82 - protein, 5 aa, synthetic construct
DRLRR

SEQ ID NO: 83 - protein, 7 aa, synthetic construct
PKQKKRK

SEQ ID NO: 84 - protein, 10 aa, synthetic construct
RKLKKKIKKL

SEQ ID NO: 85 - protein, 10 aa, synthetic construct
REKKKFLKRR

SEQ ID NO: 86 - protein, 20 aa, synthetic construct
KRKGDEVDGVDEVAKKKSKK

SEQ ID NO: 87 - protein, 17 aa, synthetic construct
RKCLQAGMNLEARKTKK

(19) United States
(12) Patent Application Publication
(10) Pub. No.: US 2025/0163474 A1
(43) Pub. Date: May 22, 2025

(54) SYSTEMS, METHODS, AND COMPOSITIONS FOR TARGETED GENE MANIPULATION AND USES THEREOF

(71) Applicant: GUANGZHOU REFORGENE MEDICINE CO., LTD., Guangzhou, Guangdong (CN)

(72) Inventors: Junbin LIANG, Guangzhou (CN); Shimin WANG, Guangzhou (CN); Hui XU, Guangzhou (CN); Huabao LIAO, Guangzhou (CN); Piao ZHANG, Guangzhou (CN); Xin LI, Guangzhou (CN); Desheng HUANGFU, Guangzhou (CN)

(73) Assignee: GUANGZHOU REFORGENE MEDICINE CO., LTD., Guangzhou, Guangdong (CN)

(21) Appl. No.: 18/956,278

(22) Filed: Nov. 22, 2024

(30) Foreign Application Priority Data
Nov. 22, 2023 (CN) 202311567658.6

(51) Int. Cl. C12N 15/90; A61K 38/00; C12N 9/22; C12N 15/11

(52) U.S. Cl. CPC C12N 15/902; C12N 9/22; C12N 15/111; A61K 38/00; C12N 2310/20

(57) ABSTRACT

A system, a method, and a composition for targeted gene manipulation and use thereof are provided. The system specifically targets an RNA molecule encoding an ApoE4 protein, which may be used to treat diseases such as Alzheimer's disease.

### CROSS-REFERENCE TO RELATED APPLICATIONS

The present application claims priority to Chinese patent application No. 202311567658.6, filed on Nov. 22, 2023, the entire contents of which are incorporated herein by reference.

### SEQUENCE LISTING

The present application contains a Sequence Listing, which has been electronically filed in XML format and is hereby incorporated herein by reference in its entirety. The XML file was created on Nov. 20, 2024, and is entitled “2024-11-22-Sequence Listing-20954-0002US00” and has a size of 104,973 bytes.

### TECHNICAL FIELD

The present disclosure belongs to the field of biomedicine, and in particular, to a system, a method, and a composition for targeted gene manipulation and use thereof.

### BACKGROUND

Human Apolipoprotein E (ApoE) is a 34 kDa glycoprotein that is abundantly expressed in the central nervous system, which is distributed in neurons, astrocytes, microglia, vascular wall cells, and other cells. After ApoE is released, cholesterol and other lipids are redistributed to neurons and plays a key role by binding to ApoE receptors on the cell surface. ApoE has three major allele variations: ApoE2, ApoE3, and ApoE4. ApoE4 is associated with an increased risk of Alzheimer (AD) and an earlier age of onset, while ApoE2 has a neuroprotective effect.

AD is a progressive neurodegenerative disease with insidious onset, which involves slow degeneration of neurons and their connections in the brain, leading to severe memory loss, intellectual disability, and decrease in motor skills and communication abilities, eventually developing into global dementia, commonly referred to as “senile dementia”, which is the most common type of dementia, accounting for 60%-80% of cases. The main pathological features of AD include 1) neuron loss; 2) β-amyloid plaques: amyloid-β (Aβ) aggregates in the extracellular space of neurons, which leads to neuronal damage and necrosis by disrupting communication between synapses; and 3) neurofibrillary tangles (NFT): hyperphosphorylated Tau protein aggregates within neuronal cells, hindering the transport of nutrients and small molecules within the neuronal cells. Currently, there is a lack of clinically effective drugs that significantly treat or prevent AD.

ApoE4 is one of the greatest risk factors for the onset of AD. Compared to the common ApoE3 allele, carrying one ApoE4 allele increases the risk of late-onset AD by 3 to 4 times, while carrying two ApoE4 alleles increases the risk of late-onset AD by 9 to 15 times. ApoE4 is also associated with an earlier age of onset of AD in patients with AD. Increasing evidence suggests that ApoE4 promotes the aggregation of A3 by inhibiting clearance of Aβ, while also contributes to the pathogenesis of AD through impairing microglial reactivity and lipid metabolism, affecting synaptic integrity and plasticity.

Preclinical animal experiments have shown that ApoE4 promotes Aβ deposition, blood-brain barrier leakage, etc. (Brain 145.10 (2022): 3582-3593.). Knockdown of ApoE4 in neurons, astrocytes, and microglia all lead to a reduction in Aβ deposition, improvement in cognitive and memory functions, and a delay in the progression of AD. (Frontiers in aging neuroscience 11(2019):14.; Journal of Neuroscience 39.37 (2019): 7408-7427.). In contrast, ApoE3, which differs from ApoE4 by only one base, plays a role in promoting synaptic integrity and regulating cholesterol levels. Existing reports indicate that the synaptic integrity in ApoE knockout mice is reduced. Notably, plasma ApoE is critical for spatial learning ability and other non-neuronal degenerative diseases (e.g., coronary heart disease). In addition, ApoE acts as an anti-inflammatory in AP-induced inflammation. ApoE antibodies have shown an elevated risk of inflammation and a slowed motor response in mice (The Journal of clinical investigation 128.5 (2018): 2144-2155. Journal of Experimental Medicine 209.12 (2012): 2149-2156.). Therefore, in the case of ApoE heterozygous patients, indiscriminately silencing of ApoE, which involves broadly reducing or eliminating ApoE expression across all cell types, may have potential side effects and should be approached with caution.

### SUMMARY

One aspect of the present disclosure provides a method for regulating the expression of at least one gene of interest. The method comprises introducing an engineered and non-naturally occurring targeting system into a eukaryotic cell containing the gene of interest, wherein the gene of interest includes at least one of an apolipoprotein E epsilon 4 (ApoE4) allele or an apolipoprotein E epsilon 3 (ApoE3) allele; and the targeting system is configured to reduce the expression level of the ApoE4 allele in the eukaryotic cell by at least 5%, or is configured to not reduce the expression level of the ApoE3 allele or reduce the expression level of the ApoE3 allele by at most 90% in the eukaryotic cell.

In some embodiments, the gene of interest includes an ApoE4 allele. In some embodiments, the gene of interest is an ApoE4 allele. In some embodiments, the gene of interest is an ApoE4 allele and an ApoE3 allele.

In some embodiments, the targeting system is configured to reduce the expression level of the APOE4 allele in the eukaryotic cell and substantially not reduce the expression level of the APOE3 allele.

In some embodiments, the method comprises introducing the engineered and non-naturally occurring targeting system into the eukaryotic cell containing the gene of interest.

An aspect of the present disclosure provides an engineered and non-naturally occurring targeting system. The targeting system is configured to regulate the expression of at least one gene of interest when introduced to a eukaryotic cell containing a gene of interest. The gene of interest includes at least one of an ApoE4 allele or an ApoE3 allele. The targeting system is configured to reduce the expression level of the ApoE4 allele in the eukaryotic cell by at least 5%, or is configured to not reduce the expression level of the ApoE3 allele or reduce the expression level of the ApoE3 allele by at most 90% in the eukaryotic cell.

In some embodiments, the gene of interest includes an ApoE4 allele. In some embodiments, the gene of interest is an ApoE4 allele. In some embodiments, the gene of interest is an ApoE4 and an ApoE3 allele.

In some embodiments, the targeting system is configured to reduce the expression level of the APOE4 allele in the eukaryotic cell and substantially not reduce the expression level of the APOE3 allele.

Another aspect of the present disclosure provides a use of a targeting system in preparing a drug for diagnosis, treatment, or prevention of Alzheimer's disease in a subject.

After contact with a cell containing an RNA molecule encoding an ApoE4 protein, the targeting system is configured to reduce a level of the RNA molecule encoding the ApoE4 protein or an ApoE4 protein in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

After contact with a cell containing an RNA molecule encoding an ApoE3 protein (ApoE3 RNA), the targeting system is configured to not reduce a level of ApoE3 RNA or the ApoE3 protein in the cell or reduce a level of the ApoE3 RNA or the ApoE3 protein in the cell by at most 95%, at most 90%, at most 85%, at most 80%, at most 75%, at most 70%, at most 65%, at most 60%, at most 55%, at most 50%, at most 45%, at most 40%, at most 35%, at most 30%, at most 25%, at most 20%, at most 15%, at most 10%, or at most 5%.

In some embodiments, the subject carries at least one ApoE4 allele.

In some embodiments, the subject carries an ApoE4 allele.

In some embodiments, the subject carries an ApoE4 allele and an ApoE3 allele.

In some embodiments, the targeting system includes, but not limited to, at least one of a gene editing system, a small interfering RNA (siRNA) system, a short hairpin RNA (shRNA) system, an antisense oligonucleotide (ASO) system, a microRNA (miRNA) system, a nucleic acid aptamer system, a CIRTS system (e.g., ORF5-TBP6.7-Pin nuclease), a LEAPER system, or a RESTORE system.

In some embodiments, the gene editing system is a CRISPR-Cas gene editing system.

In some embodiments, the CRISPR-Cas gene editing system includes, but not limited to, a CRISPR-Cas13a gene editing system, a CRISPR-Cas13b gene editing system, a CRISPR-Cas13c gene editing system, a CRISPR-Cas13d gene editing system, a dCas13-ADAR system (e.g., a ceRBE system, a REPAIR system, a RESCUE system, a CURE system), an xABE system, a mxABE system, a CRISPR-Cas12g gene editing system, a CRISPR-Cas7-11 gene editing system (e.g., Cas7-11 from Desulfonema ishimotonii), a CRISPR-Cas Csm gene editing system (e.g. type III-A CRISPR-Cas Csm complex of Thermus thermophilus), a CRISPR-CasΦ3 gene editing system, a RCas9 system (RNA-targeting Cas9).

In some embodiments, the gene editing system is a CRISPR-Cas13 gene editing system. The CRISPR-Cas13 gene editing system refers to a system that utilizes Cas13 in combination with a guide RNA (gRNA) to target and bind to a target RNA.

In some embodiments, the targeting system includes a nucleotide sequence complementary to an RNA molecule encoding an ApoE4 protein (referred to as a targeting system complementary region).

In some embodiments, the targeting system complementary region is a nucleotide sequence. Optionally, the targeting system complementary region is an RNA sequence. Optionally, the targeting system complementary region is a DNA sequence. Optionally, the targeting system complementary region is a heterozygous sequence of RNA and DNA. In some embodiments, the targeting system complementary region is a nucleotide sequence including chemical modification. The chemical modification includes, but not limited to, thiophosphate modification, 2′-OMe modification, and locked nucleic acid (LNA) modification.

In some embodiments, the targeting system includes a nucleotide sequence (referred to as a targeting system complementary region) complementary to an RNA molecule encoding an ApoE4 protein. The targeting system complementary region has an identity to any one of sequences shown in SEQ ID NO: 1 to SEQ ID NO: 54 by at least 80%, at least 85%, at least 90%, at least 95%, or 100%.

In some embodiments, the targeting system complementary region is reverse complementary to SEQ ID NO: 55 or SEQ ID NO: 56 and has one, two, or three nucleotide mismatches with SEQ ID NO: 55 or SEQ ID NO: 56. In some embodiments, the targeting system complementary region is reverse complementary to SEQ ID NO: 55 and has one, two, or three nucleotide mismatches with SEQ ID NO: 55. In some embodiments, the targeting system complementary region is reverse complementary to SEQ ID NO: 56 and has one, two, or three nucleotide mismatches with SEQ ID NO: 56. In some embodiments, the targeting system complementary region is reverse complementary to SEQ ID NO: 56 and has zero or one nucleotide mismatch with SEQ ID NO: 56. In some embodiments, the targeting system complementary region is reverse complementary to SEQ ID NO: 55 or SEQ ID NO:56, and has no nucleotide mismatch with SEQ ID NO: 55 or SEQ ID NO:56. In some embodiments, the targeting system complementary region is reverse complementary to SEQ ID NO: 55 or SEQ ID NO:56, and has only one nucleotide mismatch with SEQ ID NO: 55 or SEQ ID NO:56. In some embodiments, the targeting system complementary region is reverse complementary to SEQ ID NO: 55 or SEQ ID NO:56, and has only two nucleotide mismatches with SEQ ID NO: 55 or SEQ ID NO:56. In some embodiments, the targeting system complementary region is reverse complementary to SEQ ID NO: 55 or SEQ ID NO:56 and has only three nucleotide mismatches with SEQ ID NO: 55 or SEQ ID NO:56. Furthermore, in some embodiments, the mismatch is not located at the 32nd position of SEQ ID NO: 55 or the 21st position of SEQ ID NO: 56, or the targeting system complementary region has no mismatch at a position corresponding to a mutation site c.334T>C of the ApoE4 genotype.

In some embodiments, the targeting system complementary region has zero, one, two, or three nucleotide differences compared to any one of sequences shown in SEQ ID NO: 1 to SEQ ID NO: 8.

In some embodiments, the targeting system complementary region includes any one of sequences shown in SEQ ID NO: 1 to SEQ ID NO:54.

In some embodiments, the targeting system includes a nucleotide sequence (referred to as a targeting system complementary region) complementary to an RNA molecule encoding an ApoE4 protein. The targeting system complementary region has an identity to any one of sequences shown in SEQ ID NO: 2, SEQ ID NO: 5, SEQ ID NO: 31, SEQ ID NO: 44, and SEQ ID NO: 47 by at least 80%, at least 85%, at least 90%, at least 95%, or 100%. Optionally, in some embodiments, the targeting system complementary region is reverse complementary to SEQ ID NO: 55 or SEQ ID NO: 56, and has zero, one, two, or three nucleotide mismatches with SEQ ID NO: 55 or SEQ ID NO: 56. Optionally, the targeting system complementary region only has zero, one, two, or three nucleotide mismatches with any one of sequences shown in SEQ ID NO: 2, SEQ ID NO: 5, SEQ ID NO: 31, SEQ ID NO: 44, and SEQ ID NO: 47.

In some embodiments, the targeting system includes a nucleotide sequence (referred to as a targeting system complementary region) complementary to an RNA molecule encoding an ApoE4 protein. The targeting system complementary region has an identity to any one of sequences shown in SEQ ID NO: 2 or SEQ ID NO: 5 by at least 80%, at least 85%, at least 90%, at least 95%, or 100%. Optionally, the targeting system complementary region is reverse complementary to SEQ ID NO: 56 and has zero, one, or two nucleotide mismatches with SEQ ID NO: 56.

In some embodiments, the gene editing system includes:
a guide RNA including a guide sequence for hybridization with an RNA molecule encoding an ApoE4 protein, or a polynucleotide sequence encoding the guide RNA;
an RNA-guided nuclease, or a polynucleotide sequence encoding the nuclease;
the guide RNA is configured to form a complex with the nuclease and guide the complex to bind specifically to a sequence of the RNA molecule encoding an ApoE4 protein.

In some embodiments, the targeting system complementary region is a guide sequence of a guide RNA of the gene editing system.

In some embodiments, a gene editing system includes:
a guide RNA including a guide sequence for hybridization to an RNA molecule encoding an ApoE4 protein, or a polynucleotide sequence encoding the guide RNA; and
an RNA-guided nuclease, or a polynucleotide sequence encoding the nuclease;
the guide RNA is configured to form a complex with the nuclease and guide the complex to bind specifically to a sequence of the RNA molecule encoding an ApoE4 protein;

After contact with a cell containing an RNA molecule encoding an ApoE4 protein, the gene editing system is configured to reduce a level of the RNA molecule encoding an ApoE4 protein or an ApoE4 protein in the cell by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

After contact with a cell containing an ApoE3 RNA, the gene editing system is configured to not reduce a level of the ApoE3 RNA or an ApoE3 protein in the cell or reduce a level of the ApoE3 RNA or an ApoE3 protein in the cell by at most 95%, at most 90%, at most 85%, at most 80%, at most 75%, at most 70%, at most 65%, at most 60%, at most 55%, at most 50%, at most 45%, at most 40%, at most 35%, at most 30%, at most 25%, at most 20%, at most 15%, at most 10%, or at most 5%.

In some embodiments, the RNA molecule encoding an ApoE4 protein is mammalian ApoE4 pre-mRNA and/or ApoE4 mature mRNA.

In some embodiments of the present disclosure, the RNA molecule encoding an ApoE4 protein is human ApoE4 pre-mRNA and/or ApoE4 mature mRNA.

In some embodiments, the guide RNA guides the complex to bind and cleave the RNA molecule encoding an ApoE4 protein.

In some embodiments, the cell is a eukaryotic cell. In some embodiments, the cell is a mammalian cell. In some embodiments, the cell is a human cell.

Reduction of RNA level may be tested using conventional manners, including, but not limited to, a qPCR manner such as an RT-qPCR manner. A level of target RNA (an RNA molecule encoding ApoE4 protein or an ApoE3 RNA) in the untreated cell or cell treated with a gene editing system targeting non-mammalian genome may be tested as a negative control, and a knockdown level of target RNA in the experimental group is calculated relative to the negative control. Experimental group and negative control may be used to compare differences in the level of target RNA by editing using the same RNA-guided nuclease (e.g., nuclease guided by RNA with same expression), and gRNA containing different guide sequences. For example, the experimental group is edited using C13-2 and the described gRNA, and the negative control is edited using C13-2 and gRNA targeting bacterial genome. It is also possible to compare the experimental group to a known editing tool, e.g., a CasRx+gRNA editing tool. Reduction of RNA level may be tested using the same or similar methods as those in the embodiments of the present disclosure.

In some embodiments, a count of off-target sites when the complex binds and cleaves a target RNA (an RNA molecule encoding an ApoE4 protein or an ApoE3 RNA) is less than 40, less than 35, less than 30, less than 25, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, or less than 1. The count of off-target sites may be determined by conventional manners in the field. In some embodiments, the count of the off-target sites is determined by the intersection of the differentially expressed gene set identified by RNA sequencing and the off-target gene set predicted by the program. In some embodiments, the count of off-target sites is determined by the intersection of the downregulated differentially expressed gene set identified by RNA sequencing and the off-target gene set predicted by the program. The off-target gene may be predicted using an off-target gene prediction program known in the field with conventional parameter settings. For non-limiting examples, the program prediction method includes: using the EMBOSS-water program to perform prediction on the whole genome and full cDNA sequences of the target species (such as Homo sapiens or Mus musculus, etc.), with parameters set as gap_extend=0.5 & gap_open=10, filtering the prediction results to obtain the predicted potential target gene (including on-target gene and/or off-target gene) by comparison using both the forward and reverse strands of guide sequence of gRNA.

Conventional methods in the field may be used to test the reduction of a protein level encoded by a target RNA (an RNA molecule encoding ApoE4 protein or an ApoE3 RNA), including but not limited to ELISA and Western Blotting. Untreated cells or cells treated with a gene editing system targeting a non-mammalian genome may be used as negative control, and level of ApoE4 protein or ApoE3 protein in the experimental group may be calculated compared to the negative control. Reduction in a level of the protein encoded by target RNA may be tested using the same or similar methods as in the embodiments of the present disclosure.

In some embodiments, the guide sequence of the guide RNA has an identity to a sequence shown in SEQ ID NO: 68 (cctcgccgcggtactgcaccaggcggccgcgcacgtcctccatgtccgcgcccagccgggcc, a completely complementary sequence of SEQ ID NO: 55) by at least 80%, at least 85%, at least 90%, at least 91%, at least 92%, at least 93%, at least 94%, at least 95%, at least 96%, at least 97%, at least 98%, or 100%.

In some embodiments, the guide sequence of the guide RNA has an identity to a sequence shown in SEQ ID NO: 69 (gtactgcaccaggcggccgcgcgcacgtcctccatgtccgcgc, a completely complementary sequence of SEQ ID NO: 56) by at least 80%, at least 85%, at least 90%, at least 91%, at least 93%, at least 94%, at least 95%, at least 96%, at least 97%, at least 98%, or 100%.

In some embodiments, the guide sequence of the guide RNA has an identity to any one of sequences shown in SEQ ID NO: 1 to SEQ ID NO:54 by at least 80%, at least 85%, at least 90%, at least 91%, at least 92%, at least 93%, at least 94%, at least 95%, at least 96%, at least 97%, at least 98%, or 100%.

In some embodiments, the guide sequence of the guide RNA has an identity to any one of sequences shown in SEQ ID NO: 2, SEQ ID NO: 5, SEQ ID NO: 31, SEQ ID NO: 44, and SEQ ID NO: 47 by at least 80%, at least 85%, at least 90%, at least 91%, at least 92%, at least 93%, at least 94%, at least 95%, at least 96%, at least 97%, at least 98%, or 100%.

In some embodiments, the guide sequence of the guide RNA is reverse complementary to SEQ ID NO: 55 (ggcccggctgggcgcgcggacatggaggacgtgcgcggccgcctggtgcagtaccgcggcgagg) and only has zero, one, two, or three nucleotide mismatches with SEQ ID NO: 55 (i.e., of zero, one, two, or three pairs of complementary base mismatches). In some embodiments, the guide sequence of the guide RNA is reverse complementary to SEQ ID NO: 55, and has no nucleotide mismatch. In some embodiments, the guide sequence of the guide RNA is reverse complementary to SEQ ID NO: 55, and only has one nucleotide mismatch. In some embodiments, the guide sequence of the guide RNA is reverse complementary to SEQ ID NO: 55, and only has two nucleotide mismatches. In some embodiments, the guide sequence of the guide RNA is reverse complementary to SEQ ID NO: 55, and only has three nucleotide mismatches. Further, the mismatch is not located at the 32nd position C of SEQ ID NO: 55.

In some embodiments, the guide sequence of the guide RNA is reverse complementary to SEQ ID NO: 56 (gcgcggacatggaggacgtgcgcggccgcctggtgcagtac), and has zero, one, two, or three nucleotide mismatches. In some embodiments, the guide sequence of the guide RNA is reverse complementary to SEQ ID NO: 56, and has no nucleotide mismatch. In some embodiments, the guide sequence of the guide RNA is reverse complementary to SEQ ID NO: 56, and only has one nucleotide mismatch. In some embodiments, the guide sequence of the guide RNA is reverse complementary to SEQ ID NO: 56, and only has two nucleotide mismatches. In some embodiments, the guide sequence of the guide RNA is reverse complementary to SEQ ID NO: 56, and only has three nucleotide mismatches. Further, the mismatch is not located at the 21st position of SEQ ID NO: 56.

In some embodiments, the guide sequence of the guide RNA has an identity to any of sequences shown in SEQ ID NO: 1 to SEQ ID NO: 8 by at least 80%. In some embodiments, the guide sequence of the guide RNA has zero, one, or two nucleotide differences compared to any one of sequences shown in SEQ ID NO: 1 to SEQ ID NO: 8. In some embodiments, the guide sequence of the guide RNA has no nucleotide difference compared to any one of sequences shown in SEQ ID NO: 1 to SEQ ID NO: 8. In some embodiments, the guide sequence of the guide RNA has one nucleotide difference compared to any one of sequences shown in SEQ ID NO: 1 to SEQ ID NO: 8.

In some embodiments, the guide sequence of the guide RNA only has zero or one nucleotide difference compared to a sequence shown in SEQ ID NO: 2. In some embodiments, the guide sequence of the guide RNA only has one nucleotide difference compared to a sequence shown in SEQ ID NO: 2. Further, the difference is not located at the 13th position of SEQ ID NO: 2.

In some embodiments, the guide sequence of the guide RNA only has zero or one nucleotide difference compared to a sequence shown in SEQ ID NO: 5. In some embodiments, the guide sequence of the guide RNA only has one nucleotide difference compared to a sequence shown in SEQ ID NO: 5. Further, the difference is not located at the 19th position of SEQ ID NO: 5.

In some embodiments, the guide sequence of the guide RNA has no mismatch at a position corresponding to a mutation site c.334T>C of the ApoE4 genotype.

In some embodiments, the mismatch refers to any base pairings other than pairings of A-T, A-U, and C-G.

In some embodiments, the guide sequence of the guide RNA includes any one of sequences shown in SEQ ID NO: 1 to SEQ ID NO: 54.

In some embodiments, the guide sequence of the guide RNA includes any one of sequences shown in SEQ ID NO: 2, SEQ ID NO: 5, SEQ ID NO: 31, SEQ ID NO: 44, and SEQ ID NO: 47.

In some embodiments, the guide sequence of the guide RNA is any one of sequences shown in SEQ ID NO: 1 to SEQ ID NO: 54.

In some embodiments, the guide sequence of the guide RNA is any one of sequences shown in SEQ ID NO: 2, SEQ ID NO: 5, SEQ ID NO: 31, SEQ ID NO: 44, and SEQ ID NO: 47.

In some embodiments, the guide RNA includes a guide sequence and a scaffold sequence, and the scaffold sequence interacts with an RNA-guided nuclease. The scaffold sequence is a segment of sequence that usually remains unchanged in a guide RNA molecule when the guide RNA molecule is designed. For example, the scaffold sequence refers to a part of a guide RNA molecule other than a guide sequence. In some embodiments, the scaffold sequence is a direct repeat (DR) sequence.

In some embodiments, the RNA-guided nuclease is a Cas13 protein or a fragment thereof. The fragment of the Cas13 protein is, for example, a nucleic acid binding domain of the Cas13 protein.

In some embodiments, the Cas13 protein is a Cas13a protein, a Cas13b protein, a Cas13c protein, or a Cas13d protein. For example, LwaCas13a, LsCas13a, LbuCas13a, dLbuCas13a (R472A/H477A/R1048A/H1053A), TccCas13a, LneCas13a (LneC2c2), LbmCas13a, LbnCas13a, PpCas13a, LbfCas13a, CgCas13a, Cg2Cas13a, PspCas13b, PspCas13b H133A/H1058A, PbuCas13b, PgiCas13b, BzCas13b, RanCas13b, PguCas13b, dPguCas13b (H151A/H1121A), Cas13bt1, Cas13bt3, CcaCas13b, MisCas13b, Hgm4Cas13b, Pba4Cas13b, Bba2Cas13b, CasRx, dCasRx (R239A/H244A/R858A/H863A), CasRx_N2V8 (A134V,A140V,A141V,A143V), RspCas13d, C13-2.

In some embodiments, the Cas13 protein is a Cas13d protein.

In some embodiments, the Cas13 protein has an identity to a CasRx protein or a C13-2 protein by at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

In some embodiments, the Cas13 protein is a CasRx protein. In some embodiments, the Cas13 protein is a dCasRx with mutations in both HEPN domains (R239A and H244A in HEPN-1, R858A and H863A in HEPN-2).

In some embodiments, an amino acid sequence of the Cas13 protein has an identity to a sequence shown in SEQ ID NO: 64 by at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

In some embodiments, the Cas13 protein is a C13-2 protein. In some embodiments, the Cas13 protein is a dead C13-2 protein.

In some embodiments, the guide RNA is configured to form a complex with an RNA-guided nuclease and guide the complex to bind and cleave an RNA molecule encoding an ApoE4 protein.

In some embodiments, the guide RNA is configured to form a complex with an RNA-guided nuclease and guide the complex to bind and inhibit translation of an RNA molecule encoding an ApoE4 protein.

In some embodiments, the RNA-guided nuclease includes a protein domain.

In some embodiments, the RNA-guided nuclease includes a fused protein domain.

In some embodiments, the RNA-guided nuclease includes any one or more of the following: a subcellular localization signal, a deaminase domain, a translation activation domain, a translation inhibition domain, an RNA methylation domain, an RNA demethylation domain, a nuclease domain, a splicing factor domain, a reporter tag, and an affinity tag.

In some embodiments, the subcellular localization signal is a nuclear localization signal and/or a nuclear export signal.

In some embodiments, the RNA-guided nuclease includes a nuclear localization signal and/or a nuclear export signal.

In some embodiments, the subcellular localization signal is a mitochondrial localization signal and a chloroplast localization signal.

In some embodiments, the RNA-guided nuclease includes a nuclear localization signal and/or a nuclear export signal, and optionally a deaminase domain, a translation activation domain, or a translation inhibition domain.

In some embodiments, a polynucleotide sequence encoding an RNA-guided nuclease is linked to a regulatory sequence 1 that regulates the expression of the polynucleotide sequence, and the polynucleotide sequence encoding a guide RNA is linked to a regulatory sequence 2 that regulates the expression of the polynucleotide sequence.

In some embodiments, the regulatory sequence is a promoter sequence. In some embodiments, the regulatory sequence is an enhancer sequence. In some embodiments, the regulatory sequence is a promoter sequence and an enhancer sequence.

In some embodiments, the regulatory sequence is selected from a CMV promoter, a CMV enhancer, a CBh promoter, a U6 promoter, a brain-specific promoter, and a neural-specific promoter.

In some embodiments, at least two (e.g., two, three, four, five, six, seven, eight, nine, ten, eleven, twelve, thirteen, fourteen, fifteen, or more) guide RNAs are expressed in tandem under regulation of the same regulatory sequence. Further, the crRNA precursors (pre-cRNAs) are obtained by tandem expression, which are then processed by RNA-guided nucleases to obtain mature guide RNA molecules.

In some embodiments, the polynucleotide sequence encoding the guide RNA and the polynucleotide sequence encoding the RNA-guided nuclease are located on the same vector.

In some embodiments, the gene editing system is introduced into a cell or a cell-free system in any one of the following ways: (i) as an mRNA encoding an RNA-guided nuclease and a guide RNA, (ii) as a part of a single vector or plasmid or divided into a plurality of vectors or plasmids, (iii) as a separate RNA-guided nuclease and a guide RNA, or (iv) as an RNP complex of an RNA-guided nuclease and a guide RNA.

A second aspect of the present disclosure provides a gene editing system guide RNA (gRNA), comprising a guide sequence for hybridization with an RNA molecule encoding an ApoE4 protein.

In some embodiments, the guide sequence of the guide RNA has an identity to a sequence shown in SEQ ID NO: 68 (a completely complementary sequence of SEQ ID NO: 55) by at least 80%, at least 85%, at least 90%, at least 91%, at least 92%, at least 93%, at least 94%, at least 95%, at least 96%, at least 97%, at least 98%, or 100%.

In some embodiments, the guide sequence of the guide RNA has an identity to a sequence shown in SEQ ID NO: 69 (a completely complementary sequence of SEQ ID NO: 56) by at least 80%, at least 85%, at least 90%, at least 91%, at least 92%, at least 93%, at least 94%, at least 95%, at least 96%, at least 97%, at least 98% or 100%.

In some embodiments, the guide sequence of the guide RNA has an identity to any one of sequences shown in SEQ ID NO: 1 to SEQ ID NO: 54 by at least 80%, at least 85%, at least 90%, at least 91%, at least 92%, at least 93%, at least 94%, at least 95%, at least 96%, at least 97%, at least 98%, or 100%.

In some embodiments, the guide sequence of the guide RNA has an identity to any one of sequences shown in SEQ ID NO: 2, SEQ ID NO: 5, SEQ ID NO: 31, SEQ ID NO: 44, and SEQ ID NO: 47 by at least 80%, at least 85%, at least 90%, at least 91%, at least 92%, at least 93%, at least 94%, at least 95%, at least 96%, at least 97%, at least 98%, or 100%.

In some embodiments, the guide sequence of the guide RNA is reverse complementary to SEQ ID NO: 55, and only has one or two nucleotide mismatches. In some embodiments, the guide sequence of the guide RNA is reverse complementary to SEQ ID NO: 55, and only has one nucleotide mismatch. Further, the mismatch is not located at the 32nd position C of SEQ ID NO: 55.

In some embodiments, the guide sequence of the guide RNA is reverse complementary to SEQ ID NO: 56, and only has one or two nucleotide mismatches. In some embodiments, the guide sequence of the guide RNA is reverse complementary to SEQ ID NO: 56, and only has one nucleotide mismatch. Further, the mismatch is not located at the 21st position of SEQ ID NO: 56.

In some embodiments, the guide sequence of the guide RNA only has zero, one, or two nucleotide differences compared to any one of sequences shown in SEQ ID NO: 1 to SEQ ID NO: 8. In some embodiments, the guide sequence of the guide RNA has no nucleotide difference compared to any one of sequences shown in SEQ ID NO: 1 to SEQ ID NO: 8. In some embodiments, the guide sequence of the guide RNA only has one nucleotide difference compared to any one of sequences shown in SEQ ID NO: 1 to SEQ ID NO: 8. In some embodiments, the guide sequence of the guide RNA only has two nucleotide differences compared to any one of sequences shown in SEQ ID NO: 1 to SEQ ID NO: 8.

In some embodiments, the guide sequence of the guide RNA only has one or two nucleotide differences compared to a sequence shown in SEQ ID NO: 2. In some embodiments, the guide sequence of the guide RNA only has one nucleotide difference compared to a sequence shown in SEQ ID NO: 2. Further, the difference is not located at the 13th position of SEQ ID NO: 2.

In some embodiments, the guide sequence of the guide RNA only has one or two nucleotide differences compared to a sequence shown in SEQ ID NO: 5. In some embodiments, the guide sequence of the guide RNA only has one nucleotide difference compared to a sequence shown in SEQ ID NO: 5. Further, the difference is not located at the 19th position of SEQ ID NO: 5.

In some embodiments, the guide sequence of the guide RNA has no mismatch at a position corresponding to a mutation site c.334T>C of the ApoE4 genotype.

In some embodiments, the mismatch refers to any base pairings other than pairings of A-T, A-U, and C-G.

In some embodiments, X nucleotide mismatches refer to mismatch of X pairs of complementary bases (where X is a positive integer).

In some embodiments, the guide sequence of the guide RNA includes any one of sequences shown in SEQ ID NO: 1 to SEQ ID NO: 54.

In some embodiments, the guide sequence of the guide RNA includes any one of sequences shown in SEQ ID NO: 2, SEQ ID NO: 5, SEQ ID NO: 31, SEQ ID NO: 44, and SEQ ID NO: 47.

In some embodiments, the guide sequence of the guide RNA is any one of sequences shown in SEQ ID NO: 1 to SEQ ID NO: 54.

In some embodiments, the guide sequence of the guide RNA is any one of sequences shown in SEQ ID NO: 2, SEQ ID NO: 5, SEQ ID NO: 31, SEQ ID NO: 44, and SEQ ID NO: 47.

In some embodiments, the guide RNA includes a guide sequence and a scaffold sequence, and the scaffold sequence interacts with an RNA-guided nuclease. The scaffold sequence is a segment of sequence that usually remains unchanged in a guide RNA molecule when the guide RNA molecule is designed. For example, the scaffold sequence refers to a part of the guide RNA molecule other than the guide sequence. In some embodiments, the scaffold sequence is a direct repeat (DR) sequence.

In some embodiments, the RNA molecule encoding an ApoE4 protein is mammalian ApoE4 pre-mRNA and/or ApoE4 mature mRNA.

In some embodiments, the RNA molecule encoding an ApoE4 protein is human ApoE4 pre-mRNA and/or ApoE4 mature mRNA.

In some embodiments, the guide RNA is configured to form a complex with an RNA-guided nuclease and guide the complex to bind specifically to a sequence of the RNA molecule encoding an ApoE4 protein.

In some embodiments, the guide RNA is configured to form a complex with the RNA-guided nuclease and guide the complex to bind and cleave the RNA molecule encoding an ApoE4 protein.

In some embodiments, the complex reduces a level of the RNA molecule encoding an ApoE4 protein in a mammal (e.g., in a human).

In some embodiments, after contact with a cell containing an RNA molecule encoding an ApoE4 protein, the complex reduces the level of the RNA molecule encoding an ApoE4 protein in the cell.

In some embodiments, the complex reduces a level of the RNA molecule encoding an ApoE4 protein in a cell by at least 5%, at least 10%, at least 15%, at least 20%, at least 25%, at least 30%, at least 35%, at least 40%, at least 50%, at least 60%, at least 70%, at least 80%, at least 90%, at least 95%, at least 96%, at least 97%, at least 98%, or at least 99%. Reduction of RNA level may be tested using conventional methods in the field, including, but not limited to, a qPCR method such as an RT-qPCR method. A level of a target RNA (an RNA molecule encoding an ApoE4 protein or ApoE3 RNA) in untreated cells or cells treated with the gene editing system targeting a non-mammalian genome may be tested as a negative control. A knockdown level of a target RNA in the experimental group is then calculated relative to the negative control. Experimental group and negative control may be used to compare differences in the level of target RNA by editing using the same RNA-guided nuclease (e.g., nuclease guided by RNA with same expression), and gRNA containing different guide sequences. For example, the experimental group is edited using C13-2 and the described gRNA, and the negative control is edited using C13-2 and gRNA targeting bacterial genome. It is also possible to compare the experimental group to a known editing tool, e.g., a CasRx+gRNA editing tool. Reduction of RNA level may be tested using the same or similar methods as those in the embodiments of the present disclosure.

In some embodiments, a count of off-target sites when the complex binds and cleaves the RNA molecule encoding an ApoE4 protein is less than 40, less than 35, less than 30, less than 25, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, or less than 1. The count of off-target sites may be determined by conventional methods in the field. In some embodiments, the count of off-target sites is determined by the intersection of the differentially expressed gene set identified by RNA sequencing and the off-target gene set predicted by the program. For non-limiting examples, the program prediction method includes: using the EMBOSS-water program to perform prediction on the whole genome and full cDNA sequences of the target species (e.g., Homo sapiens or Mus musculus, etc.), with parameters set as gap_extend=0.5 & gap_open=10; and filtering the prediction results to obtain the predicted potential target gene (including on-target gene and/or off-target gene) by comparison using both the forward and reverse strands of the guide sequence of the gRNA.

In some embodiments, the complex reduces a level of protein encoded by the RNA molecule encoding an ApoE4 protein in a mammal (e.g., in a human).

In some embodiments, after contact with a cell containing a target RNA, the complex reduces a level of protein encoded by an RNA molecule encoding an ApoE4 protein in the cell.

In some embodiments, a protein encoded by the RNA molecule encoding an ApoE4 protein is an ApoE4 protein. In some embodiments, the complex reduces a level of ApoE4 protein in a cell.

In some embodiments, the complex reduces a level of ApoE4 protein in a cell by at least 5%, at least 10%, at least 15%, at least 20%, at least 25%, at least 30%, at least 35%, at least 40%, at least 50%, at least 60%, at least 70%, at least 80%, at least 90%, at least 95%, at least 96%, at least 97%, at least 98%, or at least 99%. Reduction in the level of protein encoded by the RNA molecule encoding an ApoE4 protein may be tested using conventional methods in the field, including but not limited to ELISA and Western Blotting. Untreated cells or cells treated with a gene editing system targeting a non-mammalian genome may be used as negative controls, and a knockdown level of the ApoE4 protein in the experimental group is calculated relative to the negative control group.

A third aspect of the present disclosure provides an isolated nucleic acid, wherein the isolated nucleic acid encodes a guide RNA according to any one of embodiments of the present disclosure.

A fourth aspect of the present disclosure provides a vector, comprising a polynucleotide sequence encoding a guide RNA according to any one of embodiments of the present disclosure, and a regulatory sequence for regulating the expression of the guide RNA.

In some embodiments, the vector is a viral vector. In some embodiments, the vector is an adeno-associated viral vector, an adenoviral vector, or a lentiviral vector.

In some embodiments, the regulatory sequence is a promoter sequence. In some embodiments, the regulatory sequence is an enhancer sequence. In some embodiments, the regulatory sequence is a promoter sequence and an enhancer sequence.

In some embodiments, the regulatory sequence is selected from a CMV promoter, a CMV enhancer, a CBh promoter, a U6 promoter, a brain-specific promoter, and an eye-specific promoter.

In some embodiments, the regulatory sequence is selected from a pol III promoter (e.g., U6 and H1 promoters), a pol II promoter (e.g., a retroviral Rous sarcoma virus (RSV) LTR promoter (optionally with an RSV enhancer), a cytomegalovirus (CMV) promoter (optionally with a CMV enhancer), an SV40 promoter, a dihydrofolate reductase promoter, an β-actin promoter, a phosphoglycerol kinase (PGK) promoter, or an EF1α promoter.

In some embodiments, the promoter is a constitutive promoter, which is continuously active and not regulated by external signal or molecule. Suitable constitutive promoters include, but not limited to, CMV, RSV, SV40, EF1α, CAG, and β-actin promoters. In some embodiments, the promoter is an inducible promoter regulated by an external signal or molecule (e.g., a transcription factor).

In some embodiments, the promoter is a tissue-specific promoter, which may be used to drive tissue-specific expression of RNA-guided nuclease or guide RNA. Suitable muscle-specific promoters include, but not limited to, CK8, MHCK7, myoglobin (Mb) promoter, a Desmin promoter, muscle creatine kinase (MCK) promoter and variants thereof, and a SPc5-12 synthesis promoter. Suitable immune cell-specific promoters include, but not limited to, the B29 promoter (B-cells), CD14 promoter (monocytes), CD43 promoter (leukocytes and platelets), CD68 (macrophages), and SV40/CD43 promoter (leukocytes and platelets). Suitable hematopoietic-specific promoters include, but not limited to, a CD43 promoter (leukocytes and platelets), a CD45 promoter (hematopoietic cells), INF-β (hematopoietic cells), a WASP promoter (hematopoietic cells), an SV40/CD43 promoter (leukocytes and platelets), and an SV40/CD45 promoter (hematopoietic cells). Suitable pancreas-specific promoters include, but not limited to, an elastase-1 promoter. Suitable endothelial cell-specific promoters include, but not limited to, the Fit-1 promoter and the ICAM-2 promoter. Suitable neuronal tissue/cell-specific promoters include, but not limited to, a GFAP promoter (astrocytes), a SYN1 promoter (neurons), and NSE/RU5′ (mature neurons). Suitable kidney-specific promoters include, but not limited to, an NphsI promoter (Podocyte). Suitable bone-specific promoters include, but not limited to, an OG-2 promoter (osteoblasts and odontoblasts). Suitable lung-specific promoters include, but not limited to, an SP-B promoter (lung). Suitable liver-specific promoters include, but not limited to, an SV40/Alb promoter. Suitable cardiac-specific promoters include, but not limited to, α-MHC.

In some embodiments, the promoter is a chicken β-actin (CB) promoter. The chicken (3-actin promoter may be a short chicken β-actin promoter or a long chicken β-actin promoter. In some embodiments, the promoter (e.g., the chicken β-actin promoter) includes an enhancer sequence, such as a cytomegalovirus (CMV) enhancer sequence. The CMV enhancer sequence may be a short CMV enhancer sequence or a long CMV enhancer sequence. In some embodiments, the promoter includes a long CMV enhancer sequence and a long chicken β-actin promoter. In some embodiments, the promoter includes a short CMV enhancer sequence and a short chicken β-actin promoter. However, it is known to those of skill in the art that a short CMV enhancer may be used with a long chicken β-actin promoter and that a long CMV enhancer may be used with a short chicken β-actin promoter. In some embodiments, the promoter is a CBh promoter. In some embodiments, the regulatory sequence includes an HRE enhancer element. In some embodiments, the regulatory sequence includes an NRS element and an HRE enhancer sub-element connected in series.

A fifth aspect of the present disclosure provides a vector system, comprising a polynucleotide sequence encoding a guide RNA as described in the embodiment of the present disclosure and a second regulatory sequence regulating the expression of the guide RNA, and a polynucleotide sequence encoding an RNA-guided nuclease and a first regulatory sequence regulating the expression of the RNA-guided nuclease.

In some embodiments, the vector system includes one or more vectors.

In some embodiments, the vector system comprises a plurality of vectors, the polynucleotide sequence encoding the guide RNA and the second regulatory sequence regulating the expression of the guide RNA are located on a second vector, and the polynucleotide sequence encoding an RNA-guided nuclease and the first regulatory sequence regulating the expression of the RNA-guided nuclease are located on a first vector.

In some embodiments, the regulatory sequence is a promoter sequence. In some embodiments, the regulatory sequence is an enhancer sequence. In some embodiments, the regulatory sequence is a promoter sequence and an enhancer sequence.

A sixth aspect of the present disclosure provides an adeno-associated viral (AAV) vector, comprising DNA encoding the RNA-guided nuclease and the guide RNA as described in embodiments of the present disclosure.

In some embodiments, an AAV vector includes an ssDNA genome, and the ssDNA genome includes coding sequences of an RNA-guided nuclease flanked by ITR and the guide RNA.

A seventh aspect of the present disclosure provides a lipid nanoparticle, comprising a guide RNA as described in the embodiment of the present disclosure, and an mRNA encoding an RNA-guided nuclease.

An eighth aspect of the present disclosure provides a lentiviral vector, comprising a guide RNA as described in the embodiments of the present disclosure and an mRNA encoding an RNA-guided nuclease. Optionally, the lentiviral vector is pseudotyped with an envelope protein. Optionally, the mRNA encoding an RNA-guided nuclease is linked to an aptamer sequence.

A ninth aspect of the present disclosure provides a ribonucleoprotein complex, wherein the ribonucleoprotein complex is formed from a guide RNA and an RNA-guided nuclease as described in the embodiments of the present disclosure.

A tenth aspect of the present disclosure provides a virus-like particle, comprising a ribonucleoprotein complex formed from a guide RNA and an RNA-guided nuclease described in the embodiments of the present disclosure. Optionally, the RNA-guided nuclease is fused to a gag protein.

An eleventh aspect of the present disclosure provides a eukaryotic cell, comprising a targeting system, a guide RNA, a nucleic acid, a vector, and/or a vector system as described in the embodiments of the present disclosure. Optionally, the eukaryotic cell is a mammalian cell. Optionally, the eukaryotic cell is a human cell.

In some embodiments, the eukaryotic cell includes a gene editing system as described in the embodiments of the present disclosure.

A twelfth aspect of the present disclosure provides a pharmaceutical composition, comprising a targeting system, a guide RNA, a nucleic acid, a vector, and/or a vector system as described in any one of the embodiments of the present disclosure.

In some embodiments, the pharmaceutical composition includes a gene editing system as described in any one of the embodiments of the present disclosure.

In some embodiments, the pharmaceutical composition comprises a guide RNA, a nucleic acid, a vector, and/or a vector system as described in any one of the embodiments of the present disclosure.

In some embodiments, the pharmaceutical composition comprises pharmaceutically acceptable excipients.

A thirteenth aspect of embodiments of the present disclosure provides the use of a targeting system, a guide RNA, a nucleic acid, a vector, a vector system, an adeno-associated viral vector, a lipid nanoparticle, a lentiviral vector, a ribonucleoprotein complex, a virus-like particle, a eukaryotic cell, or a pharmaceutical composition according to any of embodiments of the present disclosure in any one of the following or preparing a reagent for achieving any of the following schemes:
cleaving one or more RNA molecules encoding an ApoE4 protein or making one or more RNA molecules encoding an ApoE4 protein have nicking, activating or up-regulating one or more RNA molecules encoding an ApoE4 protein, activating or inhibiting translation of one or more RNA molecules encoding an ApoE4 protein, inactivating one or more RNA molecules encoding an ApoE4 protein, visualizing, labeling, or detecting one or more RNA molecules encoding an ApoE4 protein, binding one or more RNA molecules encoding an ApoE4 protein, transporting one or more RNA molecules encoding an ApoE4 protein, and masking one or more RNA molecules encoding an ApoE4 protein.

In some embodiments, the use of the targeting system, the guide RNA, the nucleic acid, the vector, the vector system, the adeno-associated viral vector, the lipid nanoparticle, the lentiviral vector, the ribonucleoprotein complex, the virus-like particle, the eukaryotic cell, or the pharmaceutical composition according to any of the embodiments of the present disclosure in any one of the following or preparing a reagent for achieving any of the following schemes is provided:
cleaving an RNA molecule encoding an ApoE4 protein, inhibiting translation of an RNA molecule encoding an ApoE4 protein, and binding an RNA molecule encoding an ApoE4 protein.

In some embodiments, cleavage of an RNA molecule encoding an ApoE4 protein, inhibition of translation of an RNA molecule encoding an ApoE4 protein, and binding of an RNA molecule encoding an ApoE4 protein are specific and do not affect or substantially do not affect a level of APOE2 RNA.

In some embodiments, the use of a targeting system, a guide RNA, a nucleic acid, a vector, a vector system, an adeno-associated viral vector, a lipid nanoparticle, a lentiviral vector, a ribonucleoprotein complexes, a virus-like particle, a eukaryotic cell, or a pharmaceutical composition in cleaving an RNA molecule encoding an ApoE4 protein or preparing a reagent for cleaving an RNA molecule encoding an ApoE4 protein is provided.

In some embodiments, the use of a targeting system, a guide RNA, a nucleic acid, a vector, a vector system, an adeno-associated viral vector, a lipid nanoparticle, a lentiviral vector, a ribonucleoprotein complex, a virus-like particle, a eukaryotic cell, or a pharmaceutical composition in binding an RNA molecule encoding an ApoE4 protein or preparing a reagent for binding an RNA molecule encoding an ApoE4 protein is provided.

In some embodiments, the RNA molecule encoding an ApoE4 protein is a pre-mRNA or a mature mRNA. In some embodiments, the RNA molecule encoding an ApoE4 protein is a mature mRNA. In some embodiments, the RNA molecule encoding an ApoE4 protein is a human RNA molecule encoding an ApoE4 protein. In some embodiments, the RNA molecule encoding an ApoE4 protein is human ApoE4 mRNA.

A fourteenth aspect of the present disclosure provides a method for diagnosing, treating, or preventing a disease or condition, comprising: administering an effective amount of a targeting system, a guide RNA, a nucleic acid, a vector, a vector system, an adeno-associated viral vector, a lipid nanoparticle, a lentiviral vector, a ribonucleoprotein complex, a virus-like particle, a eukaryotic cell, and/or a pharmaceutical composition of the embodiments of the present disclosure to a sample of a subject in need thereof or to a subject in need thereof.

In some embodiments, a method for diagnosing, treating, or preventing a disease or condition is provided, comprising administering an effective amount of a targeting system, a eukaryotic cell, and/or a pharmaceutical composition of embodiments of the present disclosure to a sample of a subject in need thereof to a subject in need thereof.

In some embodiments, a method for diagnosing, treating, or preventing a disease or condition is provided, comprising administering an effective amount of a gene editing system of embodiments of the present disclosure to a sample of a subject in need thereof or a subject in need thereof.

In some embodiments, the disease or condition is associated with an RNA molecule encoding an ApoE4 protein. Optionally, the disease or condition is caused by the expression of an RNA molecule encoding an ApoE4 protein.

In some embodiments, the RNA molecule encoding an ApoE4 protein is a pre-mRNA or a mature mRNA. In some embodiments, the RNA molecule encoding an ApoE4 protein is a mature mRNA. In some embodiments, the RNA molecule encoding an ApoE4 protein is a human RNA molecule encoding an ApoE4 protein. In some embodiments, the RNA molecule encoding an ApoE4 protein is a human ApoE4 mRNA.

In some embodiments, the disease or condition is Alzheimer's disease.

### BRIEF DESCRIPTION OF THE DRAWINGS

FIG. 1 is a schematic diagram illustrating the corresponding positions of screened sg1-sg8 gRNAs on a target nucleic acid.

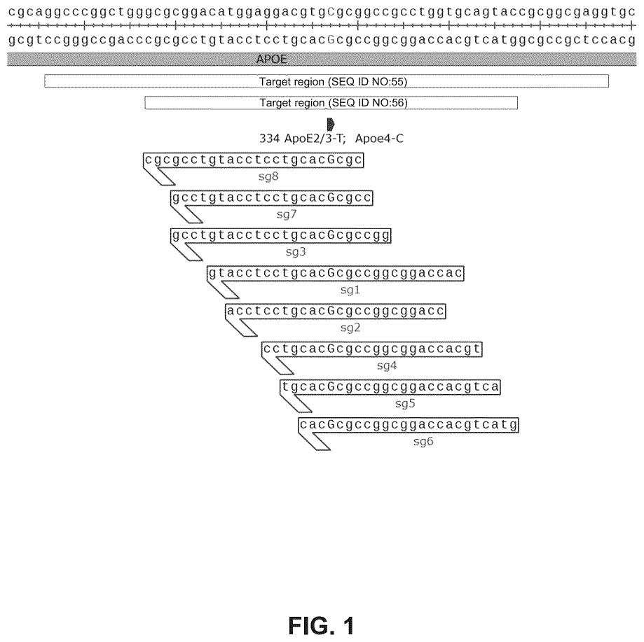

### DETAILED DESCRIPTION

The present disclosure is further illustrated below by way of embodiments, but does not thereby limit the disclosure to the scope of the described embodiments. Experimental methods that are not specified with specific conditions in the following examples should be performed according to conventional methods and conditions, or as specified in the product instructions.

### Definition

As used herein, a targeting system is a system that targets an RNA molecule encoding an ApoE4 protein. In some embodiments, the targeting system has no or little effect on an ApoE2 RNA. The targeting system includes, but not limited to, a gene editing system, a small interfering RNA (siRNA) system, a short hairpin RNA (shRNA) system, an antisense oligonucleotide (ASO) system, a microRNA (miRNA) system, a nucleic acid aptamer, a CIRTS system (e.g. ORF5-TBP6.7-Pin nuclease), a LEAPER system, and a RESTORE system.

As used herein, a term “gene editing system” refers to a protein, a nucleic acid, or combinations thereof that are configured to modify an endogenous target nucleic acid sequence when introduced into a cell. The gene editing system includes, but not limited to, a CRISPR-Cas system, a TALEN system, and a ZFN system. For example, a RNA-guided nuclease described in embodiments of the present disclosure is optionally selected from a group including Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9, Cas10, Cas10d, Cas12a/Cpf1, Cas12b/C2c1, Cas12c/C2c3, Cas12d/CasY, Cas12e/CasX, Cas12f/CasZ, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Cas13a, Cas13b, Cas13c, Cas13d, Cas13e, Cas13f, TnpB, IscB, IsrB, Fancor; or a system of fragments thereof (for example, a nucleic acid binding domain fragment).

In some embodiments, the RNA-guided nuclease is optionally selected from Cas9, Cas12, Cas13, TnpB, IscB, IsrB, a Fancor nuclease; or fragments thereof, including, but not limited to, a nucleic acid binding domain fragment.

In some embodiments, the Cas9 is selected from SpCas9, SaCas9, Nme2Cas9, Nme3Cas9, CjCas9, NmCas9, FnCas9, PpnCas9, FrCas9, SauCas9, SauriCas9, ScaCas9, St1Cas9, BlatCas9, CdiCas9, GeoCas9, fragments thereof, as well as mutants thereof or fragments of mutants thereof. In some embodiments, the RNA-guided nuclease is selected from AsCpf1 and enAsCas12a (addgene plasmid #196724), dFnCas12a (addgene plasmid #136379), ErCas12a, LbCas12a D832A, LbCas12a H759A, LbCas12a E795L, FnCas12a3, FnCas12a D917A, AsCas12a R1226A, AsCas12a D908A, AsCas12a E174R/S542R, AsCas12a (S542R/K548V/N552R), PrCas12a, PxCas12a, PcCas12a, PdCas12a, Mb2Cas12a, Mb3Cas12a, MlCas12a, CMaCas12a, CMtCas12a, HkCas12a, Lb5Cas12a, ErCas12a, TsCas12a, FnCpf1, LbCas12a, ttHsCas12a, AaCas12b, AaCas12b D570A, AaCas12b Q119F/E475R/E758R, BhCas12b, BvCas12b, BrCas12b, AkCas12b, AmCas12b, BsCas12b, OspCas12c, Cas12c2 (addgene plasmid #183072), Cas12c_4 (addgene plasmid #183071), Cas12c1 (addgene plasmid #120872), CasY.1 (from Katanobacteria), CasY.2 (from Vogelbacteria), CasY.3 (from Vogelbacteria), CasY.4 (from Parcubacteria), CasY.5 (from Komeilibacteria), CasY.6 (from Kerfeldbacteria), PlmCasX, DpbCasX, Un1Cas12f, CnCas12f1, enRhCas12f1, AsCas12f1, SpaCas12f1, Cas12g1 (addgene plasmid #120879), Cas12h disclosed in the PCT application WO2021113522A1 (SEQ ID NO: 1), Cas12i1 (addgene plasmid #171670), Cas12i2 (addgene plasmid #188275), Cas12i1 (addgene plasmid #120882), Cas12i2 (addgene plasmid #120883), Cas12i protein named Cas12f.4/Cas12f.5/Cas12f.6 disclosed in Chinese Patent Application CN111757889B, dSiCas12i (D1049A), SiCas12i, Si2Cas12i, WiCas12i, Wi2Cas12i, Wi3Cas12i, SaCas12i, Sa2Cas12i, Sa3Cas12i, WaCas12i, Wa2Cas12i, xCas12i, hfCas12Max, Cas12i-Max (addgene plasmid #188276), Cas12i1 D647A (addgene plasmid #171671), Cas12i-HiFi (addgene plasmid #188269), Cas12i1 D647A, Cas12j3 (addgene plasmid #188497), Cas12j2 (addgene plasmid #188498), AsCas12j-2 (addgene plasmid #191655), Cas12j-8 (addgene plasmid #194966), ShCas12k, N7Cas12k, AcCas12k, Cas12k-TniQ (addgene plasmid #181787), Cas12k-TnsC (addgene plasmid #181789), Cas12l, MmCas12m, MmCas12m ΔZF (H549A, C552A), dCas12m-ΔZF (D485A, H549A, C552A), AcCas12n, dAcCas12n (D240), TnpB Actinomadura_cellulosilytica_strain_DSM_45823, TnpB Actinomadura_namibiensis_strain_DSM_44197, TnpB Actinomadura_umbrina_strain_DSM_43927_$, TnpB Actinoplanes_lobatus_strain_DSM_43150 (TnpB-1 and TnpB-2), TnpB Alicyclobacillus_macrosporagiidus_strain_DSM_17980, TnpB Haloactinospora_alba_Strain_DSM_45015, TnpB Lipingzhangella_halophila_strain_DSM_102030, TnpB Meiothermus_Silvanus_DSM_9946, TnpB QNFX01000004, ISDra2 TnpB (PDB: 8H1J), KralscB-1, AwaIscB, OgeuIscB, GtFz1 (from Guillardia theta), SpuFz1 (from Spizellomyces punctatus), NlovFz2 (from Percolozoa Naegleria lovaniensis), MmeFz2 (from Mercenaria mercenaria), fragments thereof, and mutants thereof or fragments of mutants thereof.

The term “RNA-guided nuclease” refers to a polypeptide that binds to a specific sequence of a target RNA in a sequence-specific manner, and the polypeptide is guided to a target RNA by a guide RNA, and the guide RNA is complexed to the polypeptide and hybridizes to a target sequence on the target RNA. Cleavage of the target sequence by the RNA-guided nuclease may result in a strand break. Although the RNA-guided nuclease may cleave the target sequence upon binding, the term “RNA-guided nuclease” also includes anuclease-deactivated RNA-guided nuclease that can bind to, but not cleave a target sequence. The RNA-guided nuclease described in embodiments of the present disclosure include, but not limited to, a wild-type RNA-guided nuclease (e.g., C13-2, CasRx, etc.), a variant thereof (e.g., a mutant with a complete loss of cleavage activity, a mutant with a partial loss of cleavage activity, a mutant with an increased cleavage activity, a mutant with a reduced off-target effect, and a mutant with a reduced bystander effect), or functional fragments thereof or fusion proteins thereof.

As used herein, a term “Cas protein” is a CRISPR-associated (Cas) polypeptide or protein, when complexed or functionally combined with one or more guide RNAs, the Cas protein is guided to the target sequence of the target RNA and sometimes subsequently binds to or cleaves the target RNA.

In some embodiments, the gene editing system used in methods described herein is a Clustered Regularly Interspaced Short Palindromic Repeats (CRISPR)/CRISPR-associated (Cas) nuclease system, which is an engineered nuclease system based on a bacterial system that can be used for mammalian genome engineering.

As used herein, a term “knockdown” refers to a measurable reduction in a level of a target RNA in a genetically modified cell, compared to a level of a target RNA in a control cell that does not contain genetic modifications that reduce expression. For example, compared to the level of a target RNA in the control cell, the level of the target RNA in the genetically modified cell is reduced by greater than 0%, greater than or equal to 5%, greater than or equal to 10%, greater than or equal to 15%, greater than or equal to 20%, greater than or equal to 25%, greater than or equal to 30%, greater than or equal to 40%, greater than or equal to 50%, greater than or equal to 60%, greater than or equal to 70%, greater than or equal to 80%, greater than or equal to 90%, or greater than or equal to 95%. Those skilled in the art readily understand how to use gene editing-mediated inhibition techniques to knock down the target RNA or a portion thereof based on details described herein.

As used herein, terms “gene editing system guide RNA”, “guide RNA”, “guide polynucleotide”, “guide RNA”, and “gRNA” are used interchangeably. The term “guide RNA” is used to refer to a molecule in a gene editing system that forms a complex with an RNA-guided nuclease and guides the complex to bind specifically to a target sequence. The guide RNA includes a guide sequence and a scaffold sequence, and the guide sequence may hybridize with the target sequence. When the RNA-guided nuclease is a Cas protein, in particular, a Cas13 protein, the scaffold sequence is usually a direct repeat (DR) sequence.

As used herein, terms “guide sequence” and “targeting domain” are used interchangeably, which refer to a continuous nucleotide sequence in a gRNA that is partially or completely complementary to a target sequence in a target RNA and that can hybridize with the target sequence in the target RNA through base pairing facilitated by the RNA-guided nuclease. Complete complementarity of the guide sequence described herein with the target sequence is not required, as long as sufficient complementarity exists to cause hybridization and facilitate formation of a gene editing complex.

Suitable direct repeat (DR) sequences may exist in the CRISPR loci of prokaryotes (such as bacteria and archaea), the suitable DR sequences may be obtained through experimental screening, and the suitable DR sequences may also be obtained by modifying or optimizing on this basis. For non-limiting examples, deletions, substitutions, or additions of one, two, three, four, or more complementary base pairs in the complementary double-strand region of the secondary structure of the DR sequence, and deletions, substitutions, or additions of nucleotides in the loop of the stem-loop structure of the secondary structure of the DR sequence (for example, aptamer sequences may be inserted into the loop). The DR sequence has usually tens of nucleotides, some fragments of which are reverse complementary to each other, i.e., implying that a secondary structure such as a stem-loop structure (often referred to as a hairpin structure) is formed within an RNA molecule, other fragments of which manifest as unstructured. When the RNA-guided nuclease is a Cas protein, the DR sequence is a constant part of a gRNA molecule, which contains a strong secondary structure, facilitating interactions between the Cas protein and the gRNA molecule.

Terms “hybridization” or “hybridizing” refer to a process in which completely or partially complementary polynucleotide chains come together under suitable hybridization conditions to form a double-stranded structure or a region, which includes association of nucleic acids through hydrogen bonding. As used herein, the term “hybridization” includes a situation in which a double-stranded structure or a region contains one or more protrusions or mismatches. Hybridization and a strength of hybridization (i.e., a strength of association between nucleic acids) are influenced by factors such as a complementarity degree between the nucleic acids, a stringency of the involved conditions, and Tm of formed hybrids. While hydrogen bonds are generally formed between adenine and thymine, between adenine and uracil, or between cytosine and guanine, other non-classical base pairs may also form hydrogen bonds. It is to be expected that modified nucleotides may non-classically form hydrogen bonds that allow or facilitate hybridization.

As used herein, the term “target RNA” refers to a polynucleotide that contains a target sequence, denoting a specific sequence or its inverse complementary sequence thereof that one wishes to bind, target, or modify using a gene editing system. For example, the target RNA is a complete segment of a mature mRNA molecule or a pre-mRNA molecule.

As used herein, the term “target sequence” refers to a small segment of sequence of a target RNA molecule that is complementary (completely or partially) to a guide sequence of a gRNA molecule. The gene editing complex or a CRISPR complex specifically localizes to a target sequence through a guide sequence and performs a corresponding function at or near this location. A length of the target sequence is often dozens of nt (nucleotides), for example, about 10 nt, about 20 nt, about 30 nt, about 40 nt, about 50 nt, about 60 nt.

As used herein, a term “cleavage/cleaving” refers to causing a covalent bond (e.g., a covalent phosphodiester bond) in a ribose-phosphate backbone of a polynucleotide to break.

The ability of the guide RNA guiding the complex to bind specifically to a sequence of the target RNA may be assessed by any suitable manners. For example, a component of a gene editing system sufficient to form a CRISPR complex, including a guide RNA to be tested, is provided to a host cell having a corresponding target RNA molecule, through the transfection of a vector encoding the component of the CRISPR complex, the assessment of preferential cleavage within the target sequence is assessed. Similarly, cleavage of a sequence of a target RNA can be assessed in a test tube by providing the target RNA and a component of the CRISPR complex including a guide RNA to be tested and a control guide RNA different from the guide RNA for a test, and comparing the ability to bind the target RNA or a cleavage rate of the target RNA between the guide RNA to be tested and the control guide RNA. The ability of the guide RNA guiding the complex to cleave the target RNA may also be assessed by the manner described above.

As used herein, a term “identity or percent identity” is used to refer to matching of sequences between two polypeptides or between two nucleic acids. When a position in two sequences being compared is occupied by the same base or amino acid monomer subunit (e.g., a specific position in each of two DNA molecules is occupied by an adenine, or a specific position in each of two polypeptides is occupied by a lysine), then the respective molecules are identical at the position. The “percent identity” between two sequences is a function of a count of matching positions shared by the two sequences divided by a total count of positions being compared, multiplied by 100%. For example, if two sequences match in 6 out of 10 positions, then the two sequences have 60% identity. Typically, a comparison is made when comparing two sequences to produce a maximum identity. Such comparison may be performed by using published and commercially available comparison algorithms and programs, such as, but not limited to, Clustal Q, MAFFT, Probcons, T-Coffee, Probalign, and BLAST, as one of ordinary skill in the art may reasonably choose to use. The person of ordinary skill in the art can determine suitable parameters for comparing sequences, for example, any algorithms needed to achieve a superior comparison or optimal comparison for full lengths of compared sequences, and any algorithms needed to achieve a superior comparison or optimal comparison for localized portions of compared sequences.

As used herein, a term “regulatory sequence” is intended to include a promoter, an enhancer, an internal ribosome entry site (IRES), and other expression control elements (e.g., a transcription termination signal, such as a polyadenylation signal, and a poly-U sequence). Regulatory sequences include those elements that direct a continuous expression of a nucleotide sequence in various types of host cells, as well as those elements that direct the expression of a nucleotide sequence only in specific host cells (e.g., tissue-specific regulatory sequences). Tissue-specific promoters may direct the expression primarily in desired tissues of interest such as muscle, neurons, bone, skin, blood, specific organs (e.g., liver and pancreas), or cells of specific types (e.g., neuronal cells and lymphocytes). Regulatory sequences may also direct expression in a time-dependent manner such as a cell cycle-dependent or developmental stage-dependent manner, which may or may not be tissue-specific or cell type-specific. The term “regulatory sequence” also includes an enhancer element such as WPRE, a CMV enhancer, an SV40 enhancer, and an intronic sequence between exons 2 and 3 of a rabbit β-globulin. The person of skill in the art understands that the design of the expression vector may depend on factors such as the selection of the host cells to be transformed, a desired expression level, or the like. A vector may be introduced into a host cell, thereby producing an RNA-guided nuclease and/or a guide RNA described in the present disclosure.

As used herein, a term “promoter” has the meaning commonly held in the art.

As used herein, a term “enhancer promoter” has its commonly understood meaning in the art.

As used herein, in reference to nucleotide sequences encoding proteins, RNA, and CRISPR complexes/DNA/RNA, a coding sequence may be codon optimized. For example, codon optimization is performed on the encoding sequence for expression in a eukaryotic cellular environment, codon optimization is performed on the encoding sequence for expression in a mammalian cellular environment, or codon optimization is performed on the encoding sequence for expression in a human cellular environment.

As used herein, a term “codon optimization” refers to a process of altering a codon of a given gene in such a way that a polypeptide sequence encoded by the gene remains identical, and an altered codon improves the expression of the polypeptide sequence. For example, if a polypeptide is derived from a human protein sequence and expressed in E. coli, and codon optimization is performed on a DNA sequence to change human codons to codons that are more efficiently expressed in E. coli, it typically improves expression.

As used herein, a term “pharmaceutically acceptable excipient” refers to a diluent, adjuvant, drug carrier, or other excipients that is administered with an active ingredient. The choice depends on the use and the intended method of administration. The excipient should not be incompatible with the active ingredient, e.g., producing any undesired biological effect or interacting with any other component of pharmaceutical compositions in a deleterious manner. The pharmaceutical composition may be prepared by methods known in the field of pharmaceutical preparation.

When referring to an RNA sequence, “t” in the sequence is used interchangeably with “u”. When referring to a guide sequence, “t” in the sequence is used interchangeably with “u”. When referring to a direct repeat sequence, “t” in the sequence is used interchangeably with “u”.

### Targeting System

A targeting system includes, but not limited to, a gene editing system, a siRNA system, a shRNA system, an ASO system, a miRNA system, a nucleic acid aptamer, a CIRTS system (e.g. ORF5-TBP6.7-Pin nuclease), a LEAPER system, a RESTORE system.

In some embodiments, the siRNA system includes 19, 20, 21, 22, 23, or 24 base pairs. In some embodiments, two strands of the siRNA have a sequence of aggcggccgcGcacgtcctc or caggcggccgcGcacgtcctcc. In some embodiments, the two strands of the siRNA have a sequence of gcggccgcGcacgtcctcct, gcggccgcGcacgtcctcct, or aggcggccgcGcacgtcctcct.

### Guide RNA

In some embodiments, the guide RNA is configured to form a complex (which may also be referred to as a gene editing complex) with an RNA-guided nuclease and guide the complex to bind specifically to a sequence of a target RNA.

In some embodiments, the guide RNA is configured to form the complex with the RNA-guided nuclease and guide the complex to bind to and cleave the target RNA (e.g., an RNA molecule encoding an ApoE4 protein).

In some embodiments, the complex reduces a level of target RNA in a cell.

In some embodiments, the complex reduces a level of target RNA in the cell by at least 5%, at least 10%, at least 15%, at least 20%, at least 25%, at least 30%, at least 35%, at least 40%, at least 50%, at least 60%, at least 70%, at least 80%, at least 90%, at least 95%, at least 96%, at least 97%, at least 98%, or at least 99%. Reduction in a level of target RNA may be tested using conventional methods in the art, including, but not limited to, a qPCR method as described in embodiments, including: using untreated cells or cells treated with the gene editing system targeting a non-mammalian genome as a negative control, and calculating a knockdown level of target RNA in the experimental group compared to the negative control.

In some embodiments, the complex reduces a level of target RNA in the cell by at least 5%. In some embodiments, the complex reduces a level of target RNA in the cell by at least 40%. In some embodiments, the complex reduces a level of target RNA in the cell by at least 80%. In some embodiments, the complex reduces a level of target RNA in the cell by at least 85%. In some embodiments, the complex reduces the level of target RNA in the cell by at least 90%.

In some embodiments, a count of off-target sites when the complex binds to and cleaves the target RNA is less than 40, less than 35, less than 30, less than 25, less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, less than 2, or less than 1. The count of off-target sites may be determined by conventional methods in the art. In some embodiments, the count of off-target sites is determined by taking the intersection of a differentially expressed gene set determined by RNA sequencing and an off-target gene set predicted by the program. For non-limiting examples, the program prediction method includes: using the EMBOSS-water program to perform prediction on the whole genome and full cDNA sequences of the target species (such as Homo sapiens or Mus musculus, etc.), with parameters set as gap_extend=0.5 & gap_open=10, and filtering the prediction results to obtain the predicted potential target gene (including on-target gene and/or off-target gene) by comparison using both the forward and reverse strands of the guide sequence of the gRNA.

In some embodiments, the guide sequence includes 20-40 nucleotides, 20-35 nucleotides, 20-30 nucleotides, or 25-30 nucleotides.

In some embodiments, the guide sequence hybridizes with the target RNA and has nucleotide mismatches by no more than six, no more than five, no more than four, no more than three, no more than two, or no more than one.

In some embodiments, the guide sequence has an identity of 100% to a sequence of target RNA, i.e., completely complementary.

In some embodiments, the guide sequence is located either at the 3′ end or the 5′ end of the direct repeat sequence. In some embodiments, the guide sequence is located at the 3′ end of the direct repeat sequence. In some embodiments, the guide sequence is located at the 5′ end of the direct repeat sequence.

In some embodiments, the direct repeat sequence includes a sequence having an identity with a sequence shown in SEQ ID NO: 65 or SEQ ID NO: 66 by at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

In some embodiments, the direct repeat sequence includes a sequence as shown in SEQ ID NO: 65 or SEQ ID NO: 66. In some embodiments, the direct repeat sequence includes a sequence composed of a sequence shown in SEQ ID NO: 65 or SEQ ID NO: 66.

In some embodiments, the guide RNA includes an aptamer sequence.

In some embodiments, the aptamer sequence is inserted into a loop of a stem-loop structure of a secondary structure of the direct repeat sequence of the guide RNA.

In some embodiments, the guide RNA includes a modified nucleotide. Modifications include, but not limited to, 2′-O-methyl modification, 2′-O-methyl-3′-phosphorothioate modification, or 2′-O-methyl-3′-thio-PACE modification. In some embodiments, the guide RNA includes a modified nucleotide, and the modified nucleotide is selected from deoxyribonucleotides and locked nucleic acids (LNA). In some embodiments, the guide RNA includes at least one chemically modified nucleotide. In some embodiments, the guide RNA is a hybrid RNA-DNA guide, i.e., some RNA nucleotides in the guide RNA are replaced by DNA nucleotides. In some embodiments, the guide RNA is a hybrid RNA-LNA guide, i.e., some RNA nucleotides in the guide RNA are replaced by LNA nucleotides.

In some embodiments, a plurality of guide RNAs are a part of an array (which may be a part of a vector such as a viral vector or plasmid). For example, a guide array including a sequence DR-spacer region-DR-spacer region-DR-spacer region may include 3 unique and unprocessed guide RNAs (one for each DR-spacer region sequence). Once introduced into a cell or a cell-free system, the array is processed by an RNA-guided nuclease such as a Cas protein into multiple individual mature guide RNAs, which allows multiplexing such as delivering a plurality of guide RNAs to a cell or system to target a plurality of target RNAs or a plurality of regions within a single target RNA.

In some embodiments, the target RNA is located in nucleus and/or cytoplasm of a eukaryotic cell.

### Target RNA

A gene editing system and compositions described in the embodiments of the present disclosure may be used to target one or more target RNA molecules such as a target RNA molecule present in a biological sample. In some embodiments, the target RNA is a pre-mRNA or mature RNA (mRNA).

In some embodiments, the target RNA is an RNA molecule encoding an ApoE4 protein. In some embodiments, the target RNA is a human RNA molecule encoding an ApoE4 protein. In some embodiments, the target RNA is a human ApoE4 mRNA.

In some embodiments, the target RNA is an RNA molecule encoding an ApoE4 protein or a fragment thereof.

In some embodiments, the target RNA is an ApoE4 mRNA or a fragment thereof.

In some embodiments, the target RNA is ApoE4 pre-mRNA or a fragment thereof.

### RNA-Guided Nuclease

In some embodiments, the RNA-guided nuclease is a Cas protein.

In some embodiments, the RNA-guided nuclease is selected from a wild-type RNA-guided nuclease (including, but not limited to, CasRx, C13-2, etc.), a variant thereof (including, but not limited to, a mutant with complete loss of cleavage activity, a mutant with partial loss of cleavage activity, a mutant with increased cleavage activity, and a mutant with a reduced off-target/bystander effect), or functional fragments thereof or fusion proteins thereof.

In some embodiments, the RNA-guided nuclease includes any one or more of the following fusion domains (e.g., which may be referred to as fusion proteins): a subcellular localization signal, a deaminase domain, a translation activation domain, a translation inhibition domain, an RNA methylation domain, an RNA demethylation domain, a nuclease domain, a splicing factor domain, a reporter tag, and an affinity tag.

Exemplary fusion domains (e.g., fused heterologous protein domains) include domains configured to cleave an RNA (e.g., PIN endonuclease domains, NYN domains, SMR domains from SOT1, or RNase domains from staphylococcal nuclease), domains configured to affect RNA stability (e.g., tristetraprolin (TTP) or domains from UPF1, EXOSC5, and STAU1), domains configured to edit nucleotides or ribonucleotides (e.g., cytidine deaminase, PPR proteins, adenosine deaminase, ADAR-family proteins, or APOBEC-family proteins), domains configured to activate translation (e.g., eIF4E and other translation initiation factors, the yeast poly (A)-binding protein or the GLD2 domain), domains configured to inhibit translation (e.g., Pumilio or FBF PUF proteins, adenosine deaminase, CAF1, Argonaute protein), domains configured to methylate RNA (e.g., domains from m6A methyltransferase factors (e.g., METTL14, METTL3, or WTAP)), domains configured to demethylate RNA (e.g., human alkylation repair homologue 5), domains configured to affect splicing (e.g., RS-rich domain of SRSF1, Gly-rich domain of hnRNP A1, alanine-rich motif of RBM4, or proline-rich motif of DAZAP1), domains configured to achieve affinity purification or immunoprecipitation (e.g., FLAG, HA, biotin, or HALO tags), and domains configured to achieve proximity-based protein tagging and recognition (e.g., biotin ligase (e.g., BirA) or peroxidase (e.g., APEX2) to biotinylate target DNA interacting proteins).

In some embodiments, the fusion domain includes an adenosine deaminase domain. In some embodiments, anRNA-guided nuclease with a mutated HEPN domain or catalytically inactive RNA-guided nucleases are covalently linked or fused with an adenosine deaminase domain to guide A-to-I deaminase activity of RNA transcripts in mammalian cells. For example, fusing an adenosine deaminase domain engineered based on ADAR2 for targeting A-to-I RNA editing. In other embodiments, the adenosine deaminase domain is covalently linked or fused to an adaptor protein, which is configured to binding to an aptamer sequence inserted into or attached to the guide RNA, thereby allowing the adenosine deaminase domain to be non-covalently linked to an RNA-guided nuclease that is complexed with the guide RNA.

In some embodiments, the fusion domain includes a cytosine deaminase domain. In some embodiments, an RNA-guided nuclease with a mutated HEPN domain or a catalytically inactive RNA-guided nuclease is covalently linked or fused with a cytidine deaminase domain to guide C-to-U deaminase activity in RNA transcripts in mammalian cells. For example, using a cytosine deaminase domain evolved from ADAR2 for targeting C-to-U RNA editing. In other embodiments, the cytosine deaminase domain is covalently linked or fused to an adaptor protein, which is configured to bind to an aptamer sequence inserted into or attached to the guide RNA, thereby allowing the cytosine deaminase domain to be non-covalently attached to the RNA-guided nuclease that is complexed with the guide RNA.

In some embodiments, the fusion domain includes the splicing factor domain. In some embodiments, an RNA-guided nuclease with a mutated HEPN domain or a catalytically inactive RNA-guided nuclease is covalently linked or fused with a splicing factor domain to guide the alternative splicing of the target RNA in mammalian cells. For non-limiting examples, the splicing factor domains include an RS-rich domain of SRSF1, a Gly-rich domain of hnRNPA1, an alanine-rich motif of RBM4, or a proline-rich motif of DAZAPL. In other embodiments, the splicing factor domain is covalently linked or fused to an adaptor protein, which is configured to bind to an aptamer sequence inserted into or attached to the guide RNA, thereby allowing the splicing factor domain to be non-covalently attached to the RNA-guided nuclease that is complexed with the guide RNA.

In some embodiments, the fusion domain includes the translation activation domain. In some embodiments, an RNA-guided nuclease with a mutated HEPN domain or a catalytically inactive RNA-guided nuclease is covalently linked or fused with the translation activation domain to activate or enhance the expression of the target RNA. For non-limiting examples, the translation activation domains include eIF4E and other translation initiation factors, yeast poly (A)-binding protein or GLD2 domain. In other embodiments, the translation activation domain is covalently linked or fused to an adaptor protein, which is configured to bind to an aptamer sequence inserted into or attached to the guide RNA, thereby allowing the translation activation domain to be non-covalently attached to the RNA-guided nuclease that is complexed with the guide RNA.

In some embodiments, the fusion domain includes a translation inhibition domain. In some embodiments, an RNA-guided nuclease with a mutated HEPN domain or a catalytically inactive RNA-guided nuclease is covalently linked or fused with the translation inhibition domain to inhibit or decrease the expression of the target RNA. For non-limiting examples, the translation inhibition domains include Pumilio or FBF PUF proteins, adenosine deaminase, CAF1, and Argonaute protein. In other embodiments, the translation inhibition domain is covalently linked or fused to an adaptor protein, which is configured to bind to an aptamer sequence inserted into or attached to the guide RNA, thereby allowing the translation inhibition domain to be non-covalently linked to the RNA-guided nuclease that is complexed with the guide RNA.

In some embodiments, the fusion domain includes an RNA methylation domain. In some embodiments, an RNA-guided nuclease with a mutated HEPN domain or a catalytically inactive RNA-guided nuclease is covalently linked or fused with the RNA methylation domain for the methylation of the target RNA. For non-limiting examples, RNA methylation domains include m6A domain such as METTL14, METTL3, or WTAP. In other embodiments, the RNA methylation domain is covalently linked or fused to an adaptor protein, which is configured to bind to an aptamer sequence inserted into or attached to the guide RNA, thereby allowing the RNA methylation domain to be non-covalently linked to the RNA-guided nuclease that is complexed with the guide RNA.

In some embodiments, the fusion domain includes an RNA demethylation domain. In some embodiments, an RNA-guided nuclease with a mutant HEPN domain or a catalytically inactivated RNA-guided nuclease is covalently linked or fused with the RNA demethylation domain for the demethylation of the target RNA. For non-limiting examples, the RNA demethylation domains include human alkylation repair homolog 5 or ALKBH5. In other embodiments, the RNA demethylation domain is covalently linked or fused to an adaptor protein, which is configured to bind to an aptamer sequence inserted into or attached to the guide RNA, thereby allowing the RNA demethylation domain to be non-covalently attached to the RNA-guided nuclease that is complexed with the guide RNA.

In some embodiments, the fusion domain includes a ribonuclease domain. In some embodiments, the RNA-guided nuclease with a mutated HEPN domain or the catalytically inactive RNA-guided nuclease is covalently linked or fused to the ribonuclease domain to cleave the target RNA. For non-limiting examples, the ribonuclease domains include PIN endonuclease domain, NYN domain, SMR domain from SOT1, or RNase domain from staphylococcal nuclease.

In some embodiments, the fusion domain includes an affinity tag or a reporter domain. In some embodiments, an RNA-guided nuclease is covalently linked or fused to the reporter domain such as a fluorescent protein. For non-limiting examples, the reporter domain includes GST, HRP, CAT, GFP, HcRed, DsRed, CFP, YFP, BFP. In some embodiments, the RNA-guided nuclease is covalently ligated or fused to the affinity tag such as a purification tag. For non-limiting examples, the affinity tag includes HA-tag, His-tag (e.g., 6-His), Myc-tag, E-tag, S-tag, calmodulin tag, FLAG-tag, GST-tag, MBP-tag, Halo tag, or biotin.

In some embodiments, the fusion domain is located at an N end, a C end, or an N end and a C end of the RNA-guided nuclease. In some embodiments, the N end, the C end, or the N end and the C end of the RNA-guided nuclease is fused with zero, one, two, three, four, or more of the fusion domains, respectively.

In some embodiments, the RNA-guided nuclease is covalently linked to the fusion domain with or without a linkage sequence, i.e., the RNA-guided nuclease is covalently linked to the fusion domain directly (without a linkage sequence) or the RNA-guided nuclease is covalently linked to the fusion domain with a linkage sequence. Typically, the linkage sequence includes 1-100 amino acids, 1-50 amino acids, 1-30 amino acids, 1-20 amino acids, 1-10 amino acids, or 1-5 amino acids.

In some embodiments, the RNA-guided nuclease includes a subcellular localization signal.

In some embodiments, the RNA-guided nuclease includes a subcellular localization signal and a deaminase domain.

In some embodiments, the subcellular localization signal is optionally selected from a nuclear localization signal and a nuclear export signal.

In some embodiments, the RNA-guided nuclease is fused to at least one subcellular localization signal. Exemplarily, the subcellular localization signal includes an organelle localization signal such as a nuclear localization signal (NLS), a nuclear export signal (NES), or a mitochondrial localization signal.

In some embodiments, the RNA-guided nuclease is fused to at least one heterologous NLS. In some embodiments, the RNA-guided nuclease is fused to at least two NLSs. In some embodiments, the RNA-guided nuclease is fused to at least three NLSs. In some embodiments, the RNA-guided nuclease is fused to at least one N-terminal NLS and at least one C-terminal NLS. In some embodiments, the RNA-guided nuclease is fused to at least two C-terminal NLSs.

In some embodiments, the RNA-guided nuclease is fused to at least two N-terminal NLSs.

In some embodiments, the NLS is independently selected from SPKKKKRKVEAS (SEQ ID NO: 70), GPKKKRKVAAA (SEQ ID NO: 71), PKKKRKV (SEQ ID NO: 72), KRPAATKKAGQAKKKKK (SEQ ID NO: 73), PAAKRVKLD (SEQ ID NO: 74), RQRRNELKRSP (SEQ ID NO: 75), NQSSNFGPMKGGNFGGRSSGPYGGGGGQYFAKPRNQGGY (SEQ ID NO: 76), RMRIZFKNKGKDTAELRRRRVEVSVELRKAKKDEQILKRRNV (SEQ ID NO: 77), VSRKRPRP (SEQ ID NO: 78), PPKKARED (SEQ ID NO: 79), POPKKKPL (SEQ ID NO: 80), SALIKKKKKMAP (SEQ ID NO: 81), DRLRR (SEQ ID NO: 82), PKQKKRK (SEQ ID NO. 83), RKLKKKKIKKL (SEQ ID NO: 84), REKKKKFLKRR (SEQ ID NO: 85), KRKGDEVDGVDEVAKKKSKK (SEQ ID NO: 86), and RKCLQAGMNLEARKTKKK (SEQ ID NO: 87).

In some embodiments, the RNA-guided nuclease is fused to a heterologous NES. In some embodiments, the RNA-guided nuclease is fused to at least two NESs. In some embodiments, the RNA-guided nuclease is fused to at least three NESs. In some embodiments, the RNA-guided nuclease is fused to at least one N-terminal NES and at least one C-terminal NES. In some embodiments, the RNA-guided nuclease is fused to at least two C-terminal NESs.

In some embodiments, the RNA-guided nuclease is fused to at least two N-terminal NESs.

In some embodiments, the NES is independently selected from an adenovirus type 5 ElB NES, an HIV Rev NES, a MAPK NES, or a PTK2 NES.

In some embodiments, the RNA-guided nuclease is fused to the NLS and the NES, and a cleavable adaptor exists between the NLS and the NES. In some embodiments, the NES in a production cell line promotes the production of a delivery particle (e.g., a virus-like particle) containing the RNA-guided nuclease. In some embodiments, cleavage of the adaptor in a target cell exposes the NLS and promotes nuclear localization of the RNA-guided nuclease in the target cell.

In some embodiments, the RNA-guided nuclease is covalently linked to the fusion domain with or without a linkage sequence, i.e., the RNA-guided nuclease is covalently linked to the fusion domain directly (without a linkage sequence) or the RNA-guided nuclease is covalently linked the fusion domain with a linkage sequence. Typically, the linkage sequence includes 1-100 amino acids, 1-50 amino acids, 1-30 amino acids, 1-20 amino acids, 1-10 amino acids, or 1-5 amino acids.

### Cas Protein

In some embodiments, a Cas protein is a Cas9 protein, a Cas12 protein, or a Cas13 protein. In some embodiments, the Cas protein is a Cas12a protein, a Cas12b protein, a Cas12c protein, a Cas12d protein, a Cas12e protein, a Cas12f protein, a Cas12g protein, a Cas12h protein, a Cas12i protein, a Cas12j protein, and a Cas12k protein.

In some embodiments, the Cas protein includes any one or more of the following fusion domains: a subcellular localization signal, a deaminase domain, a translation activation domain, a translation inhibition domain, an RNA methylation domain, an RNA demethylation domain, a nuclease domain, a splicing factor domain, a reporter tag, and an affinity tag.

In some embodiments, the Cas protein includes the subcellular localization signal.

In some embodiments, the Cas protein includes the subcellular localization signal and the deaminase domain.

In some embodiments, the subcellular localization signal is optionally selected from a nuclear localization signal and a nuclear export signal.

In some embodiments, the Cas protein is covalently linked to the fusion domain with or without a linkage sequence, i.e., the Cas protein is covalently linked directly to the fusion domain (without a linkage sequence), or the Cas protein is covalently linked to the fusion domain with a linkage sequence. Typically, the linkage sequence includes 1-100 amino acids, 1-50 amino acids, 1-30 amino acids, 1-20 amino acids, 1-10 amino acids, or 1-5 amino acids.

### Cas13 Protein

In some embodiments, a Cas13 protein is a Cas13a protein, a Cas13b protein, a Cas13c protein, or a Cas13d protein. For example, LwaCas13a, LsCas13a, LbuCas13a, dLbuCas13a (R472A/H477A/R1048A/H1053A), TccCas13a, LneCas13a (LneC2c2), LbmCas13a, LbnCas13a, PpCas13a, LbfCas13a, CgCas13a, Cg2Cas13a, PspCas13b, PspCas13b H133A/H1058A, PbuCas13b, PgiCas13b, BzCas13b, RanCas13b, PguCas13b, dPguCas13b (H151A/H1121A), Cas13bt1, Cas13bt3, CcaCas13b, MisCas13b, Hgm4Cas13b, Pba4Cas13b, Bba2Cas13b, CasRx, dCasRx (R239A/H244A/R858A/H863A), CasRx_N2V8 (A134V, A140V, A141V, A143V), RspCas13d, and C13-2.

In some embodiments, the Cas13 protein is the Cas13d protein.

In some embodiments, the Cas13 protein has an identity to a CasRx protein or a C13-2 protein by at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

In some embodiments, the Cas13 protein is the CasRx protein. In some embodiments, the Cas13 protein is dCasRx that carries mutations in both HEPN domains (R239A or H244A of HEPN-1, and R858A or H863A of HEPN-2).

In some embodiments, an amino acid sequence of the Cas13 protein has an identity to a sequence shown in SEQ ID NO: 64 by at least 50%, at least 55%, at least 60%, at least 65%, at least 70%, at least 75%, at least 80%, at least 85%, at least 90%, at least 95%, at least 96%, at least 97%, at least 98%, at least 99%, or 100%.

In some embodiments, the Cas13 protein is a C13-2 protein. In some embodiments, the Cas13 protein is a dead C13-2 protein.

In some embodiments, the Cas13 protein includes one, two, three, four, five, or six mutations at corresponding positions of amino acid residues R210, H215, R750, H755, R785, and/or H790 of a reference protein shown in SEQ ID NO: 64. In some embodiments, the Cas13 protein has mutations A (alanine) at corresponding positions of amino acid residues R210, H215, R750, H755, R785, and/or H790 of a reference protein shown in SEQ ID NO: 64.

In some embodiments, the Cas13 protein includes mutations at corresponding positions of amino acid residues R210 and H215 of a reference protein shown in SEQ ID NO: 64. In some embodiments, the Cas13 protein includes mutations at corresponding positions of amino acid residues R750 and H755 of a reference protein shown in SEQ ID NO: 64. In some embodiments, the Cas13 protein includes mutations at corresponding positions of amino acid residues R785 and H790 of a reference protein shown in SEQ ID NO: 64.

In some embodiments, the Cas13 protein includes mutations at corresponding positions of amino acid residues R210, H215, R750, and H755 of a reference protein shown in SEQ ID NO: 64.

In some embodiments, the Cas13 protein includes mutations at corresponding positions of amino acid residues R750, H755, R785, and H790 of a reference protein shown in SEQ ID NO: 64.

In some embodiments, the Cas13 protein includes mutations at corresponding positions of amino acid residues R210, H215, R785, and/or H790 of a reference protein shown in SEQ ID NO: 64.

In some embodiments, the Cas13 protein includes mutations at corresponding positions of amino acid residues R210, H215, R750, H755, R785, and H790 of a reference protein shown in SEQ ID NO: 64.

In some embodiments, mutations at the corresponding positions of R210, R750, or R785 are A. In some embodiments, mutations at the corresponding positions of H215, H755, or H790 are A. In some embodiments, mutations at the corresponding positions of R210, H215, R750, H755, R785, and H790 are all A.

In some embodiments, the Cas13 protein is obtained by introducing a mutation in the RxxxxH motif at positions 210-215, the RxxxxH motif at positions 750-755, and/or the RxxxxH motif at positions 785-790 of a sequence shown in SEQ ID NO: 64.

In some embodiments, the Cas13 protein is obtained by introducing 1, 2, 3, 4, 5, or 6 mutations at positions R210, H215, R750, H755, R785, and/or H790 of a sequence shown in SEQ ID NO: 64. In some embodiments, the Cas13 protein is obtained by mutating to A (alanine) at positions R210, H215, R750, H755, R785, and/or H790 of a sequence shown in SEQ ID NO: 64.

In some embodiments, the Cas13 protein is obtained by mutating to A at positions R210, H215, R785, and H790 of a sequence shown in SEQ ID NO: 64. In some embodiments, the Cas13 protein is obtained by mutating to A at positions R210, H215, R750, and H755 of a sequence shown in SEQ ID NO: 64. In some embodiments, the Cas13 protein is obtained by mutating to A at positions R750, H755, R785, and H790 of a sequence shown in SEQ ID NO: 64. In some embodiments, the Cas13 protein is obtained by mutating to A at positions R210, H215, R750, H755, R785, and H790 of a sequence shown in SEQ ID NO: 64.

In some embodiments, compared to a reference protein shown in SEQ ID NO: 64, the Cas13 protein includes one or more mutations at corresponding positions of the following amino acid residues of the reference protein: R11, N34, R35, R47, R58, R63, R64, N68, N87, N265, N274, R276, R290, R294, N299, N303, R308, R314, R320, R328, N332, R341, N346, R358, N372, N383, N390, N394, R47+R290, R47+R314, R290+R314, R47+R290+R314, R308+N68, N394+N68, N87+N68, R308+N265, N394+N265, N87+N265, R308+N68+N265, N87+N68+N265, T7, A16, S260, A263, M266, N274, F288, M302, N303, L304, V305, 1311, D313, H324, P326, H327, N332, N346, T353, T360, E365, A373, M380, S382, K395, Y396, D402, D411, and 5418.

In some embodiments, the Cas13 protein is obtained by introducing any one or more mutations at following positions in a sequence shown in SEQ ID NO: 64: R11, N34, R35, R47, R58, R63, R64, N68, N87, N265, N274, R276, R290, R294, N299, N303, R308, R314, R320, R328, N332, R341, N346, R358, N372, N383, N390, N394, R47+R290, R47+R314, R290+R314, R47+R290+R314, R308+N68, N394+N68, N87+N68, R308+N265, N394+N265, N87+N265, R308+N68+N265, N87+N68+N265, T7, A16, 5260, A263, M266, N274, F288, M302, N303, L304, V305, 1311, D313, H324, P326, H327, N332, N346, T353, T360, E365, A373, M380, 5382, K395, Y396, D402, D411, and 5418.

In some embodiments, the C13-2 protein, a similar protein of C13-2 protein, a mutant of C13-2 protein, and functional fragments or fusion proteins thereof may form a complex with a direct repeat sequence including a sequence shown in SEQ ID NO: 65 or SEQ ID NO: 66 described in embodiments of the present disclosure, thereby specifically targeting the target RNA disclosed in embodiments of the present disclosure (e.g., an RNA molecule encoding the APOE4 protein) under guidance of the guide sequence.

### Nucleotide Sequence Encoding the Guide RNA or the RNA-Guided Nuclease

In some embodiments, the nucleotide sequence encoding the RNA-guided nuclease is a plasmid. In some embodiments, the nucleotide sequence encoding the RNA-guided nuclease is a portion of a viral vector genome such as a DNA genome of an AAV vector flanked by an ITR. In some embodiments, the nucleotide sequence encoding the RNA-guided nuclease is an mRNA.

In some embodiments, the nucleotide sequence encoding the guide RNA or the RNA-guided nuclease is codon optimized.

In some embodiments, the nucleotide sequence encoding the guide RNA is DNA. In some embodiments, a sequence of DNA is codon optimized.

In some embodiments, the nucleotide sequence encoding the RNA-guided nuclease is DNA or mRNA. In some embodiments, the sequence of DNA is codon optimized. In some embodiments, a sequence of mRNA is codon optimized.

In some embodiments, codon optimization is performed for expression in a desired cell type. In some embodiments, codon optimization is performed for expression in a eukaryotic cell environment. In some embodiments, codon optimization is performed for expression in a mammalian cell environment. In some embodiments, codon optimization is performed for expression in a human cell environment.

Typically, codon optimization refers to the modification of a nucleic acid sequence by replacing at least one codon (e.g., about 1, 2, 3, 4, 5, 10, 15, 20, 25, 50, or more codons) of an original sequence with codons that are more frequently used in genes of a host cell, and maintains an original amino acid sequence, to enhance expression in a target host cell. Calculation programs or algorithms to perform codon optimization are also available, e.g. codon optimization may be performed using the following tools or algorithms: ExpOptimizer, Codon OptimWiz, NGTM Codon, Codon optimization, Synthetic Gene Designer, and DNAWorks, etc.

### Vector

A vector may include any type of nucleotide, including, but not limited to, DNA and RNA, which may be single stranded or double stranded and partially obtained from a natural source, and include natural, unnatural, or altered nucleotides. Suitable vectors include those vectors designed for expression such as plasmids and viruses.

In some embodiments, a recombinant vector includes a regulatory sequence such as initiation codons and termination codons of transcription and translation, which are specific to a type of host cell (e.g., bacterial, fungal, plant, or animal) to be introduced into the vector.

In some embodiments, the recombinant vector optionally includes a gene vector element (a nucleic acid) such as a selectable tag region, a lactose manipulator, a CMV promoter, a CAG promoter, a tac promoter, a T7 RNA polymerase promoter, an SP6 RNA polymerase promoter, an SV40 promoter, an IRES sequence, a WPRE element, an ITR sequence, a FLAG-tag coding region, a c-myc tag coding region, a polyHis tag coding region, a HA tag coding region, an MBP tag coding region, a GST tag coding region, a ployA coding region, a SV40 polyadenylation signal, a SV40 replication origin, a Col E1 replication origin, a loxP site, or a Cre recombinase coding region.

### Regulatory Sequence

In some embodiments, a regulatory sequence includes one or more pol III promoters (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or any combination thereof.

### Prompter

In some embodiments, the vector includes a pol III promoter (e.g., U6 and H1 promoters), a pol II promoter (e.g., a retroviral Rous sarcoma virus (RSV) LTR promoter (optionally with an RSV enhancer), a cytomegalovirus (CMV) promoter (optionally with a CMV enhancer), an SV40 promoter, s dihydrofolate reductase promoter, a β-actin promoter, a phosphoglycerol kinase (PGK) promoter, or EF1α promoter), or a pol III promoter and a pol II promoter.

In some embodiments, the promoter is a constitutive promoter, which is continuously active and is not regulated by external signals or molecules. Suitable constitutive promoters include, but not limited to, CMV, RSV, SV40, EF1α, CAG, and β-actin promoters. In some embodiments, the promoter is an inducible promoter regulated by external signals or molecules (e.g., a transcription factor).

In some embodiments, the promoter is a tissue-specific promoter, which may be used to drive a tissue-specific expression of the Cas13 protein. Suitable muscle-specific promoters include, but not limited to, CK8, MHCK7, a myoglobin promoter (Mb), a Desmin promoter, a muscle creatine kinase (MCK) promoter and variants thereof, and a SPc5-12 synthesis promoter. Suitable immune cell-specific promoters include, but not limited to, a B29 promoter (B-cells), a CD14 promoter (monocytes), a CD43 promoter (leukocytes and platelets), a CD68 (macrophages), and an SV40/CD43 promoter (leukocytes and platelets). Suitable hematopoietic-specific promoters include, but not limited to, a CD43 promoter (leukocytes and platelets), a CD45 promoter (hematopoietic cells), INF-β (hematopoietic cells), a WASP promoter (hematopoietic cells), an SV40/CD43 promoter (leukocytes and platelets), and an SV40/CD45 promoter (hematopoietic cells). Suitable pancreas-specific promoters include, but not limited to, an elastase-1 promoter. Suitable endothelial cell-specific promoters include, but not limited to, a Fit-1 promoter and an ICAM-2 promoter. Suitable neuronal tissue/cell-specific promoters include, but not limited to, a GFAP promoter (astrocytes), a SYN1 promoter (neurons), and an NSE/RU5′ promoter (a mature neuron). Neuronal tissue/cell-specific promoters may be a GFAP promoter and a SYN1 promoter. Suitable kidney-specific promoters include, but not limited to, an NphsI promoter (podocyte). Suitable bone-specific promoters include, but not limited to, an OG-2 promoter (an osteoblast and a dentinogenic cell). Suitable lung-specific promoters include, but not limited to, a SP-B promoter (lung). Suitable liver-specific promoters include, but not limited to, an SV40/Alb promoter. Suitable cardiac-specific promoters include, but not limited to, α-MHC.

In some embodiments, the promoter is a chicken β-actin (CB) promoter. The chicken (3-actin promoter may be a short chicken β-actin promoter or a long chicken β-actin promoter. In some embodiments, the promoter (e.g., the chicken j-actin promoter) includes an enhancer sequence such as a cytomegalovirus (CMV) enhancer sequence. The CMV enhancer sequence may be a short CMV enhancer sequence or a long CMV enhancer sequence. In some embodiments, the promoter includes a long CMV enhancer sequence and a long chicken j-actin promoter. In some embodiments, the promoter includes a short CMV enhancer sequence and a short chicken j-actin promoter. However, it is known for those of skill in the art that a short CMV enhancer may be used together with a long chicken β-action promoter and that a long CMV enhancer may be used together with a short chick β-action promoter. In some embodiments, the promoter is a CBh promoter.

### Enhancer

In some embodiments, the enhancer is selected from WPRE, a CMV enhancer, an SV40 enhancer, and an intronic sequence between exons 2 and 3 of a rabbit β-globulin.

In some embodiments, the enhancer is located upstream of a promoter; however, it may also be located downstream of or within a coding sequence regulated by the promoter and remain its function. Accordingly, the enhancer, or a portion thereof, may be present in an RNA sequence transcribed from the coding sequence.

In some embodiments, the enhancer is located at 100, 200, 300, 400, 500, or more base pairs of upstream or downstream of the coding sequence regulated by the promoter.

In some embodiments, the enhancer increases the expression of the coding sequence beyond the expression level provided by the promoter.

### Adeno-Associated Viral Vector (AAV Vector)

In some embodiments, the guide RNA or the gene editing system described herein is packaged in an AAV vector, e.g., packaged into AAV1, AAV2, AAV3, AAV4, AAV5, AAV6, AAV7, AAV8, AAV9, AAV10, AAV11, AAV12, AAV13, AAV PHP.B, AAV PHP.B2, AAV PHP.B3, AAV PHP.A, AAV PHP.eB, AAV PHP.eS, AAV2.7m8, AAV8.7m8, AAV ShH10, AAVrh10, or AAVrh74 capsids.

In some embodiments, the guide RNA or the gene editing system described herein is packaged into an AAV2, AAV5, AAV6, AAV8, AAV9, or AAV PHP.eB capsids.

In some embodiments, the AAV vector described herein is optionally selected from AAV2/2, AAV2/3, AAV2/4, AAV2/5, AAV2/6, AAV2/7, AAV2/8, AAV2/9, AAV2/10, AAV2/11, AAV2/12, AAV2/13, AAV2/PHP.B, AAV2/PHP.B2, AAV2/PHP.B3, AAV2/PHP.A, AAV2/PHP.eB, AAV2/PHP.eS, AAV2/2.7m8, AAV2/8.7m8, AAV2/ShH10, AAV2/rh10, and AAV2/rh74.

In some embodiments, the AAV vector described herein is selected from AAV2/2, AAV2/5, AAV2/6, AAV2/8, AAV2/9, and AAV2/PHP.eB.

In some embodiments, the gene editing system described herein is packaged in the AAV vector, and the AAV vector includes an engineered capsid with tissue tropism such as an engineered ocular tissue-tropic capsid.

### Lipid Nanoparticle

In some embodiments, in addition to an RNA payload (an mRNA encoding an RNA-guided nuclease and a guide RNA), the lipid nanoparticle (LNP) also includes four components: cationic or ionizable lipids, cholesterol, helper lipids, and PEG-lipids. In some embodiments, the cationic or ionizable lipids include cKK-E12, C12-200, ALC-0315, DLin-MC3-DMA, DLin-KC2-DMA, FTT5, Moderna SM-102, and Intellia LPO1. In some embodiments, the PEG-lipid includes PEG-2000-C-DMG, PEG-2000-DMG, or ALC-0159. In some embodiments, the helper lipid includes DSPC.

### Lentiviral Vector

In some embodiments, the lentiviral vector is pseudotyped with a homologous or heterologous envelope protein such as VSV-G. In some embodiments, an mRNA encoding an RNA-guided nuclease is ligated to an aptamer sequence.

### Aptamer/Aptamer Sequence

In some embodiments, the guide polynucleotide further includes an aptamer sequence. In some embodiments, the aptamer sequence is inserted into a loop of the guide polynucleotide. In some embodiments, the aptamer sequence is appended to an end of the guide polynucleotide.

In some embodiments, the aptamer sequence includes an MS2 aptamer sequence, a PP7 aptamer sequence, or a QP aptamer sequence.

### Adaptor Protein

In some embodiments, the gene editing system further includes a fusion protein including an adaptor protein and a fusion domain, or a nucleic acid encoding the fusion protein, and the adaptor protein is configured to blind to the aptamer sequence.

In some embodiments, the adaptor protein includes MS2 bacteriophage coat protein (MCP), PP7 bacteriophage coat protein (PCP), or QP bacteriophage coat protein (QCP). In some embodiments, the fusion domain includes a cytosine deaminase domain, an adenosine deaminase domain, a translation activation domain, a translation inhibition domain, an RNA methylation domain, an RNA demethylation domain, a nuclease domain, a splicing factor domain, a reporter tag, and an affinity tag.

### Ribonucleoprotein (RNP) Complex

In some embodiments, the RNP complex is delivered to a eukaryotic cell, a mammalian cell, or a human cell by microinjection or electroporation. In some embodiments, the RNP complex is packaged in a virus-like particle and delivered in vivo to a mammalian or a human subject.

### Virus-Like Particle

In some embodiments, an engineered virus-like particle (VLP) is pseudotyped with homologous or heterologous envelope proteins such as VSV-G. In some embodiments, an RNA-guided nuclease is fused to a gag protein (e.g., MLVgag) via a cleavable linker, where the cleavage of the adaptor in a target cell exposes NLS located between the linker and the RNA-guided nuclease. In some embodiments, the fusion protein includes (e.g., from 5′ to 3′) a gag protein (e.g., MLVgag), one or more NES, a cleavable linker, one or more NLS, and an RNA-guided nuclease. In some embodiments, the RNA-guided nuclease is fused to a first dimerization domain, and the first dimerization domain may dimerize or heterodimerize with a second dimerization domain fused to a membrane protein, and the presence of a ligand promotes the dimerization and enriches the RNA-guided nuclease or fusion protein into the VLP.

### Eukaryotic Cell

In some embodiments, a eukaryotic cell is a mammalian cell or a human cell. In some embodiments, the eukaryotic cell is a primary eukaryotic cell, a stem cell, a tumor/cancer cell, a circulating tumor cell (CTC), a blood cell (e.g., a T cell, a B cell, an NK cell, a Tregs, etc.), a hematopoietic stem cell, a specialized immune cell (e.g., a tumor-infiltrating lymphocyte or a tumor-suppressing lymphocyte), a stromal cell in the tumor microenvironment (e.g., a cancer-associated fibroblast, etc.). In some embodiments, a cell is a brain or neuronal cell of a central or peripheral nervous system (e.g., a neuron, an astrocyte, a microglia, a retinal ganglion cell, an optic rod/cone cell, etc.).

### Disease or Condition

The literature (Li Y, Macyczko J R, Liu C C, Bu G. ApoE4 reduction: An emerging and promising therapeutic strategy for Alzheimer's disease. Neurobiol Aging. 2022 July; 115:20-28. doi: 10.1016/j.neurobiolaging.2022.03.011. Epub 2022 Mar. 22. PMID: 35453035; PMCID: PMC9133097.) revealed “ApoE reduction represents a promising therapeutic strategy for the treatment of AD patients carrying the APOE c4 allele”.

In some embodiments, a disease or condition refers to a disease or condition resulted from the expression of the target RNA, e.g., an RNA molecule encoding an ApoE4 protein.

In some embodiments, the disease or condition refers to a disease or condition resulted from an abnormally high expression of the target RNA.

In some embodiments, a pharmaceutical composition is delivered in vivo to a human subject. The pharmaceutical composition may be delivered by any effective route, and a therapeutically effective amount of the pharmaceutical composition may be delivered to a subject in need thereof. Exemplary routes of administration include, but not limited to, local administration (including, but not limited to, topical patches, creams, gels, and local liquid formulations), intravenous infusion, intravenous injection, intraperitoneal injection, intramuscular injection, intratumoral injection, subcutaneous injection, intradermal injection, intraventricular injection, intravascular injection, cerebellar injection, intravitreal injection, intravitreal injection, anterior chamber injection, tympanic injection, intranasal administration, and inhalation.

In some embodiments, a therapeutically effective amount of a gene editing system or a pharmaceutical composition described in embodiments of the present disclosure is delivered to a subject in need thereof using a suitable delivery method, which may knock down the expression of ApoE4 without reducing or barely reducing the expression of ApoE3, thereby treating, for example, diseases such as Alzheimer's disease. For example, in some embodiments, gRNA with guide sequences of sg2, sg2-23, sg5-13, and sg5-16 has a minimal off-target knockdown effect on ApoE3, while maintaining high knockdown efficiency for ApoE4, which can be used for the treatment of diseases such as Alzheimer's disease.

On the basis of conforming to the common knowledge in the field, the above-preferred conditions may be arbitrarily combined to obtain the preferred embodiments of the present disclosure.

The present disclosure is further illustrated below by way of embodiments, but does not thereby limit the present disclosure to the scope of the described embodiments. In the following embodiments, experimental methods not specified with particular conditions should be carried out according to conventional methods and conditions, or as selected based on the product instructions.

### EXAMPLES

### Example 1 Testing of Editing Efficiency

This example relates to a highly active Cas13 protein, which was newly discovered, i.e., a C13-2 protein (also known as CasRfg.4), and an amino acid sequence of which is shown in SEQ ID NO: 64.

An amino acid sequence of the C13-2 protein (SEQ ID NO: 64):

MSKDKKTKAKRMGVKALLAHGEDKLTMTTFGKGNRSKIEFTEGYHGRAL
ETPKHFGIRGFEVRRIDENVDLCGDLEEGKTIEALLVNPSEKVGEDYLK
LKGTLEKRFFGREFPHDNIRIQLIYNILDIYKILGMNVADILYALGNMQ
DTELDIDMFGQSLNNEDNLKECLKRMRPYMGYFGDIFKISPKGENIADR
EHNKKVLRCISVLRNATAHDKQDEYPWFKSSDIYETKIFKADMWKIIKD
QYREKIKKVNKDFLSKNAVNMAILFDLLNARDVEQKKQITDEFYRFTIR
KDGKNLGMNLVKIREIIIDRYASGLRDKKHDPHRQKINVIADFLIFRAL
SQNQGIIDKTVSSLRLTKDEEEKDHVYQNAAELVWGMVSNCLTPYFNDP
KNKYILKYKDAKTPGDFEDWITSKISEDDGEPFVKVLSFLCNFLEGKEI
NELLTAYIHKFECIQDFLNVISSLGENVQFQPRFALFNNASFAQNVAVQ
LRILASIGKMKPDLTEAKRPLYKAAIRMLCPPEKWEKYTSDEWLEKNML
LNSEDRKNDKKKKQVNPFRNFIAGNVIESRRFMYLVRYSKPKAVRAIMQ
NRSIVNYVLHRLPSEQVHRYASVFPENFADLEQEIDFLTKKLFEFSFEE
LLHEKDVILNNSRSHKPSLEIERLKAITGLYLSVAYIAIKNIVKANARY
YIAFAVFERDKELVKAKDARIQTKIPETDFPDYFCLTQYYLDRDEEKKF
PGDPRDKEAFFEHLRKTKRHFSKQWREWLNEKIADAKSSQATGLLLREA
RNDVEHLNVLRAIPDYIQDFRHGEKGETAMNSYFELYHYLMQRLMLKNT
ELDLSHWSGWIMRSGRPDRDLIQIAFVSLAYNLPRYRNLTKEHHFDDTV
LQKIREKESLD.

A natural direct repeat sequence corresponding to the C13-2 protein is:

(SEQ ID NO: 65)
5′-GGAAGATAACTCTACAAACCTGTAGGGTTCTGAGAC-3′

A C13-2-BsaI plasmid (sequence as shown in SEQ ID NO: 57) was obtained by work conducted by outsourcing services; the plasmid contains a codon-optimized coding sequence of the C13-2 protein (CMV-driven expression), and a coding sequence of the direct repeat sequence (U6-driven expression).

C13-2-gRNA plasmid was obtained with the following method.

Firstly, a gRNA was designed at a differential site between an ApoE3 and an ApoE4, i.e., a 334 T→C mutation site. After forward and reverse primers corresponding to a guide sequence of the gRNA were annealed, the annealed forward and reverse primers were ligated to an enzyme cutting product of the C13-2-BsaI plasmid using T4 ligase to obtain the C13-2-gRNA plasmid.

Specifically, sense and antisense strands of DNA sequence were synthesized using conventional methods. For the C13-2-BsaI plasmid, the sense strand was obtained by adding ‘agac’ to the 5′ end of the guide sequence, and the antisense strand was obtained by adding ‘aaaa’ to the 5′ end of a reverse complementary sequence of the guide sequence. The sense and antisense strands of the DNA sequence were mixed and annealed to form a double stranded DNA containing sticky ends.

The C13-2-BsaI plasmid was linearized using BsaI enzyme, and a reaction was carried out at 37° C. for 2 h. The enzyme cutting product was subjected to 1% agarose gel electrophoresis, and the gel was cut to recover the enzyme cutting product.

The enzyme cutting plasmid, the annealed primers, and DNA Ligation Kit Ver.2.1 were mixed, and incubated for 1 h at 16° C. in the PCR instrument to complete the ligation between the annealed product and the linearized backbone. After the reaction product was transformed into competent cells of E. coli Stbl3 by the reaction product, they were cultured in a medium containing corresponding antibiotics. Then colony PCR was performed to screen positive clones and extract plasmids. Finally, Sanger sequencing was used for verification, and the C13-2-gRNA plasmid was obtained, which expressed C13-2 and gRNA.

TABLE 1
Guide sequences of gRNA

| Serial | | SEQ |
|---|---|---|
| number | Guidance sequence (5′-3′) | ID NO |
| sg1 | caccaggcggccgcGcacgtcctccatg | 1 |
| sg2 | ccaggcggccgcGcacgtcctcca | 2 |
| sg3 | ggccgcGcacgtcctccatgtccg | 3 |
| sg4 | tgcaccaggcggccgcGcacgtcc | 4 |
| sg5 | actgcaccaggcggccgcGcacgt | 5 |
| sg6 | gtactgcaccaggcggccgcGcac | 6 |
| sg7 | ccgcGcacgtcctccatgtccg | 7 |
| sg8 | cgcGcacgtcctccatgtccgcgc | 8 |

A HEK293T stable transfection cell strain for overexpressing an ApoE4 was constructed.

A vector Lv-ApoE4-T2a-GFP (SEQ ID NO: 58) was constructed for overexpression of an ApoE4 gene and an EGFP gene. ApoE4 was spaced from EGFP using 2A peptide. An Lv-ApoE4-T2a-GFP plasmid-packaged lentivirus was transduced into 293T cells to form a stable cell line for overexpressing an ApoE4 gene.

### Detection of Editing Efficiency

The C13-2-gRNA plasmid was transfected into the HEK293T stable transfection cell strain for overexpressing an ApoE4 (for negative control, using the C13-2-BsaI plasmid for transfection) by strictly following the procedure for the use of Life Tech's Lipofectamine 2000 (Thermo Fisher) reagent. One day before transfection, the HEK293T stable transfection cell for overexpressing an ApoE4 in good condition was inoculated into 24-well plate at 1×10⁵/well. On the day of transfection, 500 ng of the corresponding volume of recombinant gRNA plasmid was added to 50 μl of OPTIMEM (Thermo), and 1 μl of Lipofactamine 2000 was added to 50 μl of OPTIMEM medium, and then the two were mixed well and left at room temperature for 5 min. After that, a diluted plasmid DNA was mixed with Lipofactamine 2000 and left at room temperature for 15 min, then 100 μl of plasmid was added into each well of the 24-well plate, and the plate was gently shaken to mix. The cells were placed at 37° C. for culture for 72 h.

Total RNA from 293T cells was extracted using the Aikore Universal RNA Extraction Kit. Then, gDNA digestion and reverse transcription were performed using the Evo M-MLV Reverse Transcription Kit, resulting in the corresponding cDNA. The Q-PCR reaction was performed using the SYSB Green Pro Taq HS Premix qPCR Kit for detection, with GAPDH as the internal control, to obtain a level of RNA molecule of a cleaved RNA molecule encoding an ApoE4 protein for each group. Three replicates were performed in parallel for per primer pair of per sample in Q-PCR. Q-PCR primers were shown in Table 2 below.

A relative expression amount of the RNA molecule encoding an ApoE4 protein after knockdown was obtained by the 2-AACT calculation method, and the relative value was subtracted from 1 to obtain an editing efficiency value (i.e., knockdown efficiency), and an average was taken for each group. Results were shown in Table 3 below.

TABLE 2
Primers used for Q-PCR

| Primer name | Sequence (5′→3′) | Serial number |
|---|---|---|
| ApoE4-F | CGGACATGGAGGACGTGC | SEQ ID NO: 59 |
| ApoE4-R | CTGGTACACTGCCAGGCG | SEQ ID NO: 60 |
| GAPDH-F | CCATGGGGAAGGTGAAGGTC | SEQ ID NO: 61 |
| GAPDH-R | GAAGGGGTCATTGATGGCAAC | SEQ ID NO: 62 |

TABLE 3
Knockdown efficiency of RNA molecule encoding ApoE4 protein

| Groups | Guide sequence (5′-3′) | Knockdown efficiency/% |
|---|---|---|
| sg1 | caccaggcggccgcGcacgtcctccatg | 53.81 |
| sg2 | ccaggcggccgcGcacgtcctcca | 85.06 |
| sg3 | ggccgcGcacgtcctccatgtccg | 46.97 |
| sg4 | tgcaccaggcggccgcGcacgtcc | 40.82 |
| sg5 | actgcaccaggcggccgcGcacgt | 86.2 |
| sg6 | gtactgcaccaggcggccgcGcac | 55.6 |
| sg7 | ccgcGcacgtcctccatgtccg | 49.21 |
| sg8 | cgcGcacgtcctccatgtccgcgc | 56.32 |

As can be seen from the data in the table, editing activities of gRNAs with guide sequences of sg2 and sg5 are significantly higher.

### Example 2 Mismatch Effects on Non-Specific Targeting of an ApoE3

sg2 and sg5 were selected, and a single mutation was introduced at each position of a guide sequence of gRNA to obtain a new guide sequence, as shown in Table 4.

C13-2-gRNA plasmid expressing the gRNA containing the new guide sequence was obtained by constructing using a method similar to that of Example 1:

After the forward and reverse primers corresponding to the guide sequence of the gRNA were annealed, the annealed forward and reverse primers were ligated to an enzyme cutting product of the C13-2-BsaI plasmid using T4 ligase to obtain the C13-2-gRNA plasmid.

Specifically, sense and antisense strands of DNA sequence were synthesized using conventional methods. For the C13-2-BsaI plasmid, the sense strand is obtained by adding ‘agac’ to the 5′ end of the guide sequence, and the antisense strand is obtained by adding ‘aaaa’ to the 5′ end of a reverse complementary sequence of the guide sequence. The sense and antisense strands of the DNA sequence were mixed and annealed to form a double stranded DNA containing sticky ends.

The C13-2-BsaI plasmid was linearized using BsaI enzyme, and a reaction was carried out at 37° C. for 2 h. The enzyme cutting product obtained was subjected to 1% agarose gel electrophoresis, and the gel was cut to recover the enzyme cutting product.

The enzyme cutting plasmid, the annealed primer, and DNA Ligation Kit Ver.2.1 were mixed, and incubated for 1 h at 16° C. in the PCR instrument to complete the ligation between the annealed product and the linearized backbone. After the reaction product was transformed into competent cells of E. coli Stb13, they were cultured in a medium containing corresponding antibiotics. Then colony PCR was performed to screen positive clones and extract plasmids. Finally, sanger sequencing was used for verification, and the C13-2-gRNA plasmid was obtained.

TABLE 4
Related sequences of gRNA

| Serial | | |
|---|---|---|
| number | Guide sequence (5′-3′) | SEQ ID NO |
| sg2-1 | tcaggcggccgcGcacgtcctcca | 9 |
| sg2-2 | ctaggcggccgcGcacgtcctcca | 10 |
| sg2-3 | cctggcggccgcGcacgtcctcca | 11 |
| sg2-4 | ccatgcggccgcGcacgtcctcca | 12 |
| sg2-5 | ccagtcggccgcGcacgtcctcca | 13 |
| sg2-6 | ccaggtggccgcGcacgtcctcca | 14 |
| sg2-7 | ccaggctgccgcGcacgtcctcca | 15 |
| sg2-8 | ccaggcgtccgcGcacgtcctcca | 16 |
| sg2-9 | ccaggcggtcgcGcacgtcctcca | 17 |
| sg2-10 | ccaggcggctgcGcacgtcctcca | 18 |
| sg2-11 | ccaggcggcctcGcacgtcctcca | 19 |
| sg2-12 | ccaggcggccgtGcacgtcctcca | 20 |
| sg2-13 | ccaggcggccgcGtacgtcctcca | 21 |
| sg2-14 | ccaggcggccgcGctcgtcctcca | 22 |
| sg2-15 | ccaggcggccgcGcatgtcctcca | 23 |
| sg2-16 | ccaggcggccgcGcacttcctcca | 24 |
| sg2-17 | ccaggcggccgcGcacgacctcca | 25 |
| sg2-18 | ccaggcggccgcGcacgttctcca | 26 |
| sg2-19 | ccaggcggccgcGcacgtcttcca | 27 |
| sg2-20 | ccaggcggccgcGcacgtccacca | 28 |
| sg2-21 | ccaggcggccgcGcacgtccttca | 29 |
| sg2-22 | ccaggcggccgcGcacgtcctcta | 30 |
| sg2-23 | ccaggcggccgcGcacgtcctcct | 31 |
| sg5-1 | tctgcaccaggcggccgcGcacgt | 32 |
| sg5-2 | attgcaccaggcggccgcGcacgt | 33 |
| sg5-3 | acagcaccaggcggccgcGcacgt | 34 |
| sg5-4 | acttcaccaggcggccgcGcacgt | 35 |
| sg5-5 | actgtaccaggcggccgcGcacgt | 36 |
| sg5-6 | actgctccaggcggccgcGcacgt | 37 |
| sg5-7 | actgcatcaggcggccgcGcacgt | 38 |
| sg5-8 | actgcactaggcggccgcGcacgt | 39 |
| sg5-9 | actgcacctggcggccgcGcacgt | 40 |
| sg5-10 | actgcaccatgcggccgcGcacgt | 41 |
| sg5-11 | actgcaccagtcggccgcGcacgt | 42 |
| sg5-12 | actgcaccaggtggccgcGcacgt | 43 |
| sg5-13 | actgcaccaggctgccgcGcacgt | 44 |
| sg5-14 | actgcaccaggcgtccgcGcacgt | 45 |
| sg5-15 | actgcaccaggcggtcgcGcacgt | 46 |
| sg5-16 | actgcaccaggcggctgcGcacgt | 47 |
| sg5-17 | actgcaccaggcggcctcGcacgt | 48 |
| sg5-18 | actgcaccaggcggccgtGcacgt | 49 |
| sg5-19 | actgcaccaggcggccgcGtacgt | 50 |
| sg5-20 | actgcaccaggcggccgcGctcgt | 51 |
| sg5-21 | actgcaccaggcggccgcGcatgt | 52 |
| sg5-22 | actgcaccaggcggccgcGcactt | 53 |
| sg5-23 | actgcaccaggcggccgcGcacga | 54 |

A vector Lv-ApoE3-T2a-GFP (SEQ ID NO: 63) was constructed for an overexpress of an ApoE3 gene and an EGFP gene. ApoE3 was spaced from EGFP using a 2A peptide. An Lv-ApoE3-T2a-GFP plasmid-packaged lentivirus was transduced into 293T cells to form a stable cell line for overexpressing the ApoE3 gene.

### Detection of Editing Efficiency

The C13-2-gRNA plasmid was transfected into HEK293T cells overexpressing ApoE4 and HEK293T cells overexpressing ApoE3, respectively (for negative control, using the C13-2-BsaI plasmid for transfection) by strictly following procedure for the use of Life Tech's Lipofectamine 2000 (Thermno Fisher) reagent. One day before transfection, the HEK293T cells overexpressing ApoE4 and HEK293T cells overexpressing ApoE3 in good condition were inoculated into the 24-well plate at 1×10⁵/well, respectively. On the day of transfection, 500 ng of the corresponding volume of recombinant gRNA plasmid was added to 50 μl of OPTI-MEM (Thermo), and 1 μl of Lipofactamine 2000 was added to 50 μl of OPTIMEM medium, and the two were mixed well and left at room temperature for 5 min. After that, a diluted plasmid DNA was mixed with Lipofactamine 2000 and left at room temperature for 15 min, then 100 μl of plasmid was added into each well of the 24-well plate, and the plate was gently shaken to mix. The cells were placed at 37° C. for culture for 72 h.

After extracting total RNA of 293T cells using the Aikore Universal RNA Extraction Kit, gDNA digestion and reverse transcription were performed using the Evo M-MLV Reverse Transcription Kit to obtain the corresponding cDNA. The Q-PCR reaction was performed using the SYSB Green Pro Taq HS premix qPCR kit for detection, and GAPDH was used as an internal reference to obtain levels of RNA molecules of the cleaved RNA encoding an ApoE4 and the cleaved RNA encoding ApoE3 for each group. Three replicates were performed in parallel for per primer pair of per sample in Q-PCR. Q-PCR primers were shown in Table 5 below.

A relative expression amount of the RNA molecule encoding an ApoE4 or an ApoE3 was calculated by the 2-AACT calculation method, and the relative value was subtracted from 1 to obtain an editing efficiency value (i.e., knockdown efficiency). An average was taken for each group. The results were shown in Table 6 below.

TABLE 5
Primers used for Q-PCR of ApoE 293T cells

| Primer name | Sequence (5′→3′) | Serial number |
|---|---|---|
| ApoE4-F | CGGACATGGAGGACGTGC | SEQ ID NO: 59 |
| ApoE4-R | CTGGTACACTGCCAGGCG | SEQ ID NO: 60 |
| ApoE3-F | CGGACATGGAGGACGTGT | SEQ ID NO: 67 |
| ApoE3-R | CTGGTACACTGCCAGGCG | SEQ ID NO: 60 |
| GAPDH-F | CCATGGGGAAGGTGAAGGTC | SEQ ID NO: 61 |
| GAPDH-R | GAAGGGGTCATTGATGGCAAC | SEQ ID NO: 62 |

TABLE 6
Knockdown efficiency of RNA molecules encoding ApoE3 protein/ApoE4 protein

| | | Knockdown | Knock down |
|---|---|---|---|
| Serial | | efficiency of | efficiency of |
| number | Guide sequence (5′-3′) | ApoE/% | ApoE3/% |
| sg2 | ccaggcggccgcGcacgtcctcca | 82 | 22 |
| sg2-1 | tcaggcggccgcGcacgtcctcca | 59 | 74 |
| sg2-2 | ctaggcggccgcGcacgtcctcca | no knockdown | no knockdown |
| sg2-5 | ccagtcggccgcGcacgtcctcca | 39 | no knockdown |
| sg2-6 | ccaggtggccgcGcacgtcctcca | 37 | no knockdown |
| sg2-8 | ccaggcgtccgcGcacgtcctcca | no knockdown | no knockdown |
| sg2-10 | ccaggcggctgcGcacgtcctcca | no knockdown | no knockdown |
| sg2-12 | ccaggcggccgtGcacgtcctcca | no knockdown | no knockdown |
| sg2-14 | ccaggcggccgcGctcgtcctcca | 14 | no knockdown |
| sg2-15 | ccaggcggccgcGcatgtcctcca | 1 | no knockdown |
| sg2-16 | ccaggcggccgcGcacttcctcca | 6 | no knockdown |
| sg2-17 | ccaggcggccgcGcacgacctcca | 17 | no knockdown |
| sg2-18 | ccaggcggccgcGcacgttctcca | 19 | no knockdown |
| sg2-19 | ccaggcggccgcGcacgtcttcca | 50 | no knockdown |
| sg2-20 | ccaggcggccgcGcacgtccacca | 10 | 7 |
| sg2-21 | ccaggcggccgcGcacgtccttca | 19 | 16 |
| sg2-23 | ccaggcggccgcGcacgtcctcct | 75 | 7 |
| sg5 | actgcaccaggcggccgcGcacgt | 54 | 58 |
| sg5-1 | tctgcaccaggcggccgcGcacgt | untested | 87 |
| sg5-3 | acagcaccaggcggccgcGcacgt | 63 | 79 |
| sg5-6 | actgctccaggcggccgcGcacgt | 70 | 70 |
| sg5-7 | actgcatcaggcggccgcGcacgt | 74 | 64 |
| sg5-8 | actgcactaggcggccgcGcacgt | 58 | 74 |
| sg5-9 | actgcacctggcggccgcGcacgt | 43 | 62 |
| sg5-10 | actgcaccatgcggccgcGcacgt | 26 | 73 |
| sg5-11 | actgcaccagtcggccgcGcacgt | 63 | 65 |
| sg5-13 | actgcaccaggctgccgcGcacgt | 86 | 24 |
| sg5-14 | actgcaccaggcgtccgcGcacgt | 78 | 50 |
| sg5-16 | actgcaccaggcggctgcGcacgt | 74 | no knockdown |
| sg5-18 | actgcaccaggcggccgtGcacgt | 88 | 85 |
| sg5-19 | actgcaccaggcggccgcGtacgt | 77 | 78 |
| sg5-21 | actgcaccaggcggccgcGcatgt | 72 | 82 |
| sg5-23 | actgcaccaggcggccgcGcacga | 75 | 52 |

As can be seen from data in the table, gRNAs with guide sequences of sg2, sg2-23, sg5-13, and sg5-16 have a small effect on the non-specific knockdown of ApoE3, while maintaining a high knockdown efficiency for ApoE4.

Although specific embodiments of the present disclosure have been described above, those skilled in the art will understand that these are merely exemplary, and that various modifications or alterations can be made to these embodiments without departing from the principles and spirit of the present disclosure.

1. A method for regulating the expression of at least one gene of interest, comprising: introducing an engineered and non-naturally occurring targeting system into a eukaryotic cell containing the gene of interest; wherein
the gene of interest includes at least one of an apolipoprotein E epsilon 4 (ApoE4) allele or an apolipoprotein E epsilon 3 (ApoE3) allele; and
the targeting system is configured to reduce the expression level of the ApoE4 allele in the eukaryotic cell by at least 5%, or is configured to not reduce the expression level of the ApoE3 allele or reduce the expression level of the ApoE3 allele by at most 90% in the eukaryotic cell.

2. The method of claim 1, wherein the targeting system comprises at least one of a gene editing system, a small interfering RNA (siRNA) system, a short hairpin RNA (shRNA) system, an antisense oligonucleotide system, a microRNA system, a nucleic acid aptamer system, a CIRTS system, a LEAPER system, or a RESTORE system.

3. The method of claim 2, wherein the gene editing system is a CRISPR-Cas13 gene editing system.

4. The method of claim 1, wherein the targeting system includes a targeting system complementary region that is complementary to an RNA molecule encoding an ApoE4 protein, and the targeting system complementary region is a nucleotide sequence;
the targeting system complementary region is reverse complementary to SEQ ID NO: 55 or SEQ ID NO: 56 and has one, two, or three nucleotide mismatches with SEQ ID NO: 55 or SEQ ID NO: 56, and the mismatch is not located at the 32nd position of SEQ ID NO: 55 or the 21st position of SEQ ID NO: 56; or
the targeting system complementary region is reverse complementary to SEQ ID NO: 55 or SEQ ID NO: 56 and has no mismatch with SEQ ID NO: 55 or SEQ ID NO: 56.

5. The method of claim 1, wherein the targeting system includes a targeting system complementary region that is complementary to an RNA molecule encoding an ApoE4 protein, the targeting system complementary region is a nucleotide sequence, and the targeting system complementary region has an identity to any one of sequences shown in SEQ ID NO: 1 to SEQ ID NO: 54 by at least 80%.

6. The method of claim 1, wherein the targeting system is a gene editing system, and the gene editing system includes:
a guide RNA including a guide sequence for hybridization with an RNA molecule encoding an ApoE4 protein, or a polynucleotide sequence encoding the guide RNA; and
a nuclease, or a polynucleotide sequence encoding the nuclease;
wherein the guide RNA is configured to form a complex with the nuclease and guide the complex to bind specifically to a sequence of the RNA molecule encoding the ApoE4 protein, and the guide sequence of the guide RNA has an identity to any one of sequences shown in SEQ ID NO: 1 to SEQ ID NO: 54, SEQ ID NO. SEQ ID NO: 68, and SEQ ID NO: 69 by at least 80%.

7. The method of claim 6, wherein the guide sequence of the guide RNA has zero, one, two, or three nucleotide differences compared to any one of sequences shown in SEQ ID NO: 1 to SEQ ID NO: 8.

8. The method of claim 7, wherein the difference is not located at the 13th position of SEQ ID NO: 2 and/or the 19th position of SEQ ID NO: 5.

9. The method of claim 6, wherein the guide RNA includes the guide sequence and a scaffold sequence, and the scaffold sequence interacts with the nuclease.

10. The method of claim 6, wherein the nuclease is a Cas13 protein or a fragment thereof.

11. The method of claim 6, wherein the nuclease includes one or more of: a subcellular localization signal, a deaminase domain, a translation activation domain, a translation inhibition domain, an RNA methylation domain, an RNA demethylation domain, a nuclease domain, a splicing factor domain, a reporter tag, and an affinity tag.

12. The method of claim 6, wherein the polynucleotide sequence encoding the nuclease is linked to a first regulatory sequence regulating the expression of the polynucleotide sequence encoding the nuclease, and the polynucleotide sequence encoding the guide RNA is linked to a second regulatory sequence regulating the expression of the polynucleotide sequence encoding the guide RNA.

13. An engineered and non-naturally occurring targeting system, wherein
the targeting system is configured to regulate the expression of at least one gene of interest when introduced into a eukaryotic cell containing the gene of interest; wherein the gene of interest includes at least one of an apolipoprotein E epsilon 4 (ApoE4) allele or an apolipoprotein E epsilon 3 (ApoE3) allele; and
the targeting system is configured to reduce the expression level of the ApoE4 allele in the eukaryotic cell by at least 5% or is configured to not reduce the expression level of the ApoE3 allele or reduce the expression level of the ApoE3 allele by at most 90% in the eukaryotic cell.

14. The targeting system of claim 13, comprising a targeting system complementary region that is complementary to an RNA molecule encoding an ApoE4 protein, the targeting system complementary region being a nucleotide sequence; wherein
the targeting system complementary region is reverse complementary to SEQ ID NO: 55 or SEQ ID NO: 56 and has one, two, or three nucleotide mismatches with SEQ ID NO: 55 or SEQ ID NO: 56, and the mismatch is not located at the 32nd position of SEQ ID NO: 55 or at the 21st position of SEQ ID NO: 56; or
the targeting system complementary region is reverse complementary to SEQ ID NO: 55 or SEQ ID NO: 56 and has no mismatch with SEQ ID NO: 55 or SEQ ID NO: 56.

15. The targeting system of claim 13, comprising a targeting system complementary region that is complementary to an RNA molecule encoding an ApoE4 protein, wherein the targeting system complementary region is a nucleotide sequence, and the targeting system complementary region has an identity to any one of sequence shown in SEQ ID NO: 1 to SEQ ID NO: 54 by at least 80%.

16. The targeting system of claim 13, comprising at least one of a gene editing system, a siRNA system, a shRNA system, an antisense oligonucleotide system, a microRNA system, a nucleic acid aptamer system, a CIRTS system, a LEAPER system, or a RESTORE system.

17. The targeting system of claim 13, wherein the targeting system is a gene editing system, and the gene editing system includes:
a guide RNA including a guide sequence for hybridization with an RNA molecule encoding an ApoE4 protein, or a polynucleotide sequence encoding the guide RNA; and
a nuclease, or a polynucleotide sequence encoding the nuclease;
wherein the guide RNA is configured to form a complex with the nuclease and guide the complex to bind specifically to a sequence of the RNA molecule encoding the ApoE4 protein, the guide sequence of the guide RNA has an identity to any one of sequences shown in SEQ ID NO: 1 to SEQ ID NO: 54, SEQ ID NO. SEQ ID NO:
68, and SEQ ID NO: 69 by at least 80%.

18. The targeting system as claimed in claim 17, wherein the nuclease is a Cas13 protein or a fragment thereof, and the Cas13 protein is a Cas13a protein, a Cas13b protein, a Cas13c protein, or a Cas13d protein.

19. The targeting system of claim 17, wherein the nuclease is a Cas13 protein, and the Cas13 protein has an identity with CasRx or a sequence shown in SEQ ID NO: 64 by at least 50%.

20. A pharmaceutical composition, comprising the targeting system of claim 13, formulated with a pharmaceutically acceptable excipient.
